(19) United States
(12) Patent Application Publication
(10) Pub. No.: US 2023/0172830 A1
(43) Pub. Date: Jun. 8, 2023

(54) COMPOSITION COMPRISING AT LEAST ONE ALKOXYSILANE OF FORMULA (I), AT LEAST ONE ALKOXYSILANE OF FORMULA (II), AT LEAST ONE NONAMINO SILICONE, AND OPTIONALLY PIGMENTS AND/OR DIRECT DYES

(71) Applicant: L’OREAL, Paris (FR)

(72) Inventors: Zhibo LIU, AULNAY-SOUS-BOIS (FR); Andrew GREAVES, AULNAY-SOUS-BOIS (FR); Anne-Claude DUBLANCHET, AULNAY-SOUS-BOIS (FR)

(21) Appl. No.: 17/637,315

(22) PCT Filed: Aug. 21, 2020
(86) PCT No.: PCT/EP2020/073465

§ 371 Date: Feb. 22, 2022

(30) Foreign Application Priority Data
Aug. 22, 2019 (FR) FR1909346

(51) Int. Cl. A61K 8/58; A61K 8/893; A61K 8/892; A61Q 5/06

(52) U.S. Cl. CPC A61K 8/585; A61K 8/892; A61K 8/893; A61Q 5/065; A61K 2800/43; A61K 2800/882

(57) ABSTRACT

The present invention relates to a composition (F) for treating keratin fibers, notably human keratin fibers such as the hair, comprising at least one alkoxysilane of formula (I), at least one alkoxysilane of formula (II), at least one nonamino silicone of formula (III), optionally at least one coloring agent chosen from pigments, direct dyes and mixtures thereof, and optionally water.

The present invention relates to a composition (F) for treating keratin fibers, notably human keratin fibers such as the hair, comprising at least one alkoxysilane of formula (I), at least one alkoxysilane of formula (II), at least one nonamino silicone of formula (III), optionally at least one coloring agent chosen from pigments, direct dyes and mixtures thereof, and optionally water.

The present invention also relates to a process for treating keratin fibers, notably human keratin fibers such as the hair, using a mixture of two compositions (A) and (B) and said mixture being applied to the keratin fibers.

The present invention also relates to a multi-compartment device that is suitable for performing the process. The present invention also covers a process for dyeing keratin fibers.

### TECHNICAL FIELD

It is known practice to perform processes for treating keratin fibers in order notably to improve the conditioning of the hair. These processes generally consist in applying to the keratin fibers hair compositions comprising polymers that are cationic or protonatable depending on the pH, in high concentrations.

These hair compositions do, admittedly, have conditioning power, but this power may remain insufficient for good shaping and repair of the hair, or may make the keratin fibers heavy, for example making them coarser, with an unpleasant feel, and more difficult to style.

In addition, since the fibers may appear coarse and difficult to comb, this may cause damage which may lead to breakage of said fibers. Keratin fibers may also bear static electricity which disrupts their styling, their manageability and the hairstyle hold in the course of the day, the hair strands being unmanageable and poorly aligned.

There is thus a real need to develop compositions for treating keratin fibers, which are capable of maintaining or even improving the quality of the fiber such as the softness, the feel, the manageability, the smoothness, the disentangling, the sheen, the sensitivity to humidity, the reduction of frizziness and the strength of the hair strands, for example by reducing the breakage of the hair strands.

It is also known practice to use for treating keratin fibers, in particular human keratin fibers such as the hair, polymers notably used as film-forming agents. However, these compounds are not always satisfactory since they may make the hair strand heavy and dull, provide tackiness in particular in a humid environment, or even make the keratin fibers difficult to style.

Moreover, in the field of dyeing keratin fibers, in particular human keratin fibers, it is already known practice to dye keratin fibers via various techniques using direct dyes for non-permanent dyeing, or dye precursors for permanent dyeing.

Non-permanent dyeing or direct dyeing consists in dyeing keratin fibers with dye compositions containing direct dyes. They are applied to the keratin fibers for a time necessary to obtain the desired coloring, and are then rinsed out.

The standard dyes that are used are, in particular, dyes of the nitrobenzene, anthraquinone, nitropyridine, azo, xanthene, acridine, azine or triarylmethane type, or natural dyes.

Some of these dyes may be used under lightening conditions, which enables the production of colorings that are visible on dark hair.

Another dyeing method consists in using pigments. Specifically, the use of pigment on the surface of keratin fibers generally makes it possible to obtain visible colorings on dark hair, since the surface pigment masks the natural color of the fiber. These colorings obtained via this dyeing method may have the drawback of having poor resistance to water and/or shampoo washing and also to external agents such as sebum, perspiration, and mechanical actions such as blow-drying and/or rubbing. The colorings obtained may also generate staining and/or transfer, in particular when the fibers are wet.

Furthermore, compositions for temporarily dyeing the hair may also lead to a hair feel that is uncosmetic and/or not natural; the hair thus dyed may notably lack softness and/or suppleness and/or strand separation.

In order to improve the transfer-resistance, hold and/or water-resistance properties of a composition on keratin materials, it is known practice to use a hybrid material consisting of a partially or totally crosslinked polymer network obtained by hydrolysis and condensation of one or more organometallic precursors by sol-gel reaction in the presence of at least one organic polymer as described in patent FR 1 221 929.

A “sol-gel” process is a process involving the partial or total hydrolysis and then the condensation of the ingredients of a starting solution. Such technologies used in hair dyeing may have the drawback of having poor resistance to shampoo washing and to external attacking factors.

The need thus remains for a composition for treating keratin fibers, notably the hair, which has the advantage of producing a homogeneous and smooth, colored or uncolored coating on the hair, maintaining complete strand separation of the hair, which is easy to style, and limiting the sensitivity to humidity such as reducing the frizziness, while at the same time forming a coating that is persistent with respect to shampoo washing and to the various external attacking factors to which the hair may be subjected, such as blow-drying and/or rubbing, without degrading the hair.

Thus, the aim of the present invention is to develop a composition for treating keratin fibers, notably human keratin fibers such as the hair, which has the advantage of producing a homogeneous and smooth, colored or uncolored coating on the hair, said coating producing complete strand separation of the hair and making it easy to style, while at the same time forming a coating that is persistent with respect to shampoo washing and to the various attacking factors to which the hair may be subjected, such as blow-drying and/or rubbing, without degrading the hair and improving the sensitivity to humidity such as reducing the frizziness.

### Presentation of the Invention

One subject of the present invention is thus a composition (F) for treating keratin fibers, notably human keratin fibers such as the hair, comprising:
a) at least one alkoxysilane chosen from the compounds of formula (I), oligomers thereof and/or mixtures thereof as described below;
b) at least one alkoxysilane chosen from the compounds of formula (II), oligomers thereof and/or mixtures thereof as described below;
c) at least one nonamino silicone of formula (III) as described below;
d) optionally at least one coloring agent chosen from pigments, direct dyes and mixtures thereof; and
e) optionally water.

The present invention also relates to a process for treating human keratin fibers such as the hair, which consists in extemporaneously mixing, at the time of use, two compositions (A) and (B) and in applying the mixture, composition (F) as described previously, to the fibers, with:
composition (A) comprising at least one alkoxysilane chosen from the compounds of formula (I), oligomers thereof and/or mixtures thereof as described below and at least one alkoxysilane chosen from the compounds of formula (II), oligomers thereof and/or mixtures thereof; as described below, and optionally water;
composition (B) comprising at least one nonamino silicone of formula (III) as described below;
it being understood that composition (A) and/or composition (B) optionally comprise at least one coloring agent chosen from pigments, direct dyes and mixtures thereof.

The present invention also relates to a device for treating human keratin fibers such as the hair, comprising two compartments containing:
in a first compartment (E1), a composition (A) comprising at least one alkoxysilane chosen from the compounds of formula (I), oligomers thereof and/or mixtures thereof as described below and at least one alkoxysilane chosen from the compounds of formula (II), oligomers thereof and/or mixtures thereof as described below, and optionally water;
in a second compartment (E2), a composition (B) comprising at least one nonamino silicone of formula (III) as described below;
it being understood that composition (A) and/or composition (B) optionally comprise at least one coloring agent chosen from pigments, direct dyes and mixtures thereof.

By using this composition (F), colored or uncolored coatings are obtained on the hair, leading to hydrophobization of the keratin fibers, which are persistent with respect to shampoo washing. When composition (F) contains at least one coloring agent, the use of composition (F) leads to a coloring that is visible on all types of hair, said coloring being persistent with respect to shampoo washing and preserving the physical qualities of the keratin fibers, without impairing the integrity of the treated fibers. Such a coating may be resistant to the external attacking factors to which the hair may be subjected, such as blow-drying and/or sensitivity to humidity such as perspiration. It makes it possible in particular to obtain a smooth, uniform and hydrophobic deposit.

Moreover, this composition (F) gives hair with complete strand separation, which can be styled without any problem. The hair after treatment with composition (F) can be subjected to shaping treatments, preferably temporary shaping treatments.

For the purposes of the present invention, the term “alkoxysilane” means an alkoxysilane or an alkoxysilane oligomer of formula (I) or of formula (II).

The term “hair with strand separation” means hair which, after application of the composition and drying, is not stuck together (or of which all the strands are separated from each other) and thus does not form clumps of hair.

For the purposes of the present invention, the term “coloring that is persistent with respect to shampoo washing” means that the coloring obtained persists after one shampoo wash, preferably after three shampoo washes.

The term “including a” should be understood as meaning “including at least one”, unless otherwise specified.

The term “at least one” means one or more.

The invention is not limited to the examples illustrated. The features of the various examples may notably be combined within variants which are not illustrated.

For the purposes of the present invention and unless otherwise indicated:
the term “alkyl” denotes a linear or branched saturated hydrocarbon-based radical containing, for example, from 1 to 20 carbon atoms;
the term “aminoalkyl” denotes an alkyl radical as defined previously, said alkyl radical being substituted with an amino NH₂ group;
the term “hydroxyalkyl” denotes an alkyl radical as defined previously, said alkyl radical being substituted with a hydroxyl OH group;
an “alkylene” radical denotes a linear or branched saturated divalent C₂-C₄ hydrocarbon-based group such as methylene, ethylene or propylene;
a “cycloalkyl” radical denotes a saturated cyclic hydrocarbon-based group comprising from 1 to 3 rings, preferably 1 to 2 rings, more preferentially 1 ring, and comprising from 3 to 20 carbon atoms, preferably between 5 and 10 carbon atoms, such as cyclopentyl, cyclohexyl, cycloheptyl, norbornyl or isobornyl, the cycloalkyl radical possibly being substituted with one or more (C₁-C₄)alkyl groups such as methyl;
an “aryl” radical is an unsaturated cyclic aromatic radical comprising from 6 to 12 carbon atoms, which is mono- or bicyclic, fused or nonfused; preferably, the aryl group consists of 1 ring and contains 6 carbon atoms, such as phenyl;
the term “alkoxy” denotes an “alkyl-oxy” group with “alkyl” as defined previously.

Composition (F) for treating keratin fibers, notably human keratin fibers such as the hair, according to the invention is preferably a composition for dyeing human keratin fibers such as the hair.

### Alkoxysilane of Formula (I)

The composition according to the invention comprises at least one alkoxysilane chosen from the compounds of formula (I) below, oligomers thereof and/or mixtures thereof:

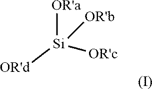

in which:
R′a, R′b, R′c and R’d, which may be identical or different, represent an alkyl group containing from 1 to 10 carbon atoms, preferably from 1 to 6 carbon atoms, more preferentially from 1 to 4 carbon atoms, such as 2 carbon atoms.

The term “oligomer” means compound(s) including at least two silicon atoms, obtained by oligomerization or polymerization of the compounds of formula (I).

Preferably, R′a, R′b, R′c and R’d are identical and denote an ethyl radical.

Among the alkoxysilanes of formula (I), the oligomers thereof and/or mixtures thereof, mention may notably be made of tetraethoxysilane (TEOS), tetrapropoxysilane, tetrabutoxysilane, tetrapentoxysilane and tetrahexyloxysilane.

Preferably, the alkoxysilane(s) of formula (I), the oligomers thereof and/or mixtures thereof are chosen from tetraethoxysilane (TEOS), tetrapropoxysilane, tetrabutoxysilane, tetrapentoxysilane and tetrahexyloxysilane and mixtures thereof, more preferentially tetraethoxysilane (TEOS).

TEOS may be purchased, for example, from the company Evonik under the name Dynasylan® A or Dynasylan® A SQ, or from the company Sigma-Aldrich under the reference 333859.

Composition (F) according to the invention may comprise one or more alkoxysilanes of formula (I), oligomers thereof and/or mixtures thereof present in a total amount ranging from 0.1% to 30% by weight, preferably from 0.5% to 25% by weight and better still from 0.5% to 20% by weight, relative to the total weight of composition (F).

### Alkoxysilane of Formula (II)

The composition according to the invention comprises at least one alkoxysilane chosen from the compounds of formula (II) below, oligomers thereof and/or mixtures thereof:

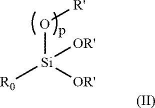

in which:
Rₒ represents an alkyl group containing from 4 to 24 carbon atoms, optionally interrupted with a dialkylammonium divalent radical RₐR_{b}N⁺An⁻with Rₐ and R_{b} independently representing an alkyl group containing from 1 to 10 carbon atoms, in particular from 1 to 4 carbon atoms, and An- representing a cosmetically acceptable anion which ensures the electrical neutrality of the compounds of formula (II), notably halide such as fluoride, chloride, bromide or iodide and more particularly chloride or bromide;
R′, which may be identical or different, represents a hydrogen atom; an alkyl group containing from 1 to 20 carbon atoms, preferably from 1 to 6 carbon atoms, more preferentially from 1 to 4 carbon atoms;
p denotes an integer ranging from 0 to 1.

The term “oligomer” means compound(s) including at least two silicon atoms, obtained by oligomerization or polymerization of the compounds of formula (II).

Preferably, Rₒ represents an alkyl group containing from 4 to 24 carbon atoms, optionally interrupted with a dialkylammonium divalent radical RₐR_{b}N⁺An⁻with Rₐ, R_{b} and An-as defined previously, R′ are identical and represent an alkyl group containing from 1 to 4 carbon atoms such as an ethyl, and p is equal to 1.

More preferentially, Rₒ represents an alkyl group containing from 6 to 18 carbon atoms, in particular 8, 12 or 16 carbon atoms and most particularly 8 carbon atoms, R′ are identical and represent an alkyl group containing from 1 to 4 carbon atoms such as an ethyl, and p is equal to 1.

According to a particular embodiment of the invention, Rₒ represents an alkyl group containing from 4 to 24 carbon atoms interrupted with a dialkylammonium divalent radical RₐR_{b}N⁺An⁻with Rₐ and R_{b} denoting a C₁-C₂ alkyl radical such as methyl, An- as defined previously, R′ are identical and represent an alkyl group containing from 1 to 4 carbon atoms such as an ethyl, and p is equal to 1.

According to this particular embodiment of the invention, Rₒ represents an alkyl group containing from 20 to 22 carbon atoms interrupted with a dialkylammonium divalent radical An⁻ with An- as defined previously, R′ are identical and represent an alkyl group containing from 1 to 4 carbon atoms such as an ethyl, and p is equal to 1.

Among the alkoxysilane compounds of formula (II), mention may be made of n-octyltriethoxysilane sold by the company Sigma-Aldrich under the reference 440213 or dodecyltriethoxysilane sold by the company Sigma-Aldrich under the reference 44237, hexadecyltrimethoxysilane sold by the company Sigma-Aldrich under the reference 52360.

Among the cationic alkoxysilane compounds of formula (II), mention may be made of dimethyloctadecyl[3-(trimethoxysilyl)propyl]ammonium chloride sold by Sigma-Aldrich under the reference 435694.

According to a preferred embodiment, the alkoxysilane of formula (II) is n-octyltriethoxysilane.

Composition (F) according to the invention may comprise one or more alkoxysilanes of formula (II), oligomers thereof and/or mixtures thereof present in a total amount ranging from 0.1% to 30% by weight, preferably from 0.5% to 25% by weight, better still from 0.5% to 20% by weight and even better still from 0.5% to 10% by weight, relative to the total weight of composition (F).

### Nonamino Silicone of Formula (III)

The composition according to the invention comprises at least one nonamino silicone of formula (III) below:

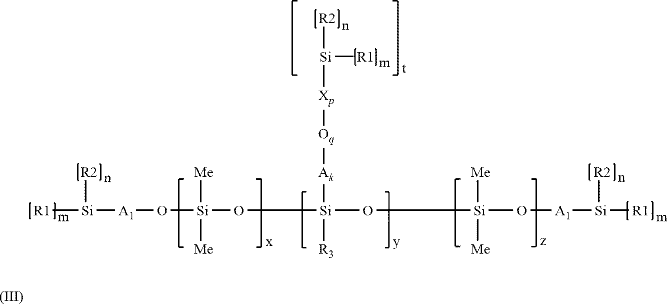

in which:
R1 independently represents a hydroxyl group or an alkoxy group containing from 1 to 2 carbon atoms or an alkyl group containing from 1 to 2 carbon atoms;
R2 independently represents an alkyl group containing from 1 to 20 carbon atoms, preferably from 1 to 10 carbon atoms and more particularly from 1 to 4 carbon atoms, such as methyl or ethyl;
R3 represents a hydroxyl group; an alkyl group containing from 1 to 10 carbon atoms, notably from 1 to 4 carbon atoms, said alkyl group being optionally substituted with at least one group chosen from a hydroxyl group (OH) or a thiol group (SH); a cycloalkyl group containing from 3 to 20 carbon atoms, notably from 5 to 6 carbon atoms, said cycloalkyl being optionally substituted with at least one group chosen from a hydroxyl group (OH) or a thiol group (SH); an alkoxy group containing from 1 to 2 carbon atoms, optionally substituted with at least one group chosen from a hydroxyl group (OH) or a thiol group (SH); an aryl group containing from 6 to 12 carbon atoms, optionally substituted with at least one group chosen from a hydroxyl group (OH) or a thiol group (SH); or a radical -(O)p--Si(R₁)_{m′}(R₂)_{n′};
A represents a saturated divalent hydrocarbon-based radical containing from 1 to 2 carbon atoms,
X represents a hydrogen atom or a saturated divalent hydrocarbon-based radical containing from 1 to 2 carbon atoms;
m denotes an integer ranging from 1 to 3, n denotes an integer ranging from 0 to 2 and m+n = 3;
m′ denotes an integer ranging from 1 to 3, n′ denotes an integer ranging from 0 to 2 and m′+n′ = 3;
p is an integer equal to 0, 1, 2 or 3; p′ is an integer equal to 0 or 1;
-k is an integer equal to 0, 1, 2 or 3;
q denotes an integer equal to 0 or 1;
j denotes an integer equal to 0 or 1;
t denotes an integer equal to 0 or 1;
y denotes an integer ranging from 0 to 10, z denotes an integer ranging from 0 to 500 with x+z ranging from 0 to 500 and x+y+z ≥ 4; and
it being understood that if X represents a hydrogen atom, then t = 0 and p = 1 and if p = 0, then t = 1 and q = 1 and that at least one of the radicals R₁ or R₃ denotes a hydroxyl radical or an alkoxy radical containing from 1 to 2 carbon atoms.

The term “nonamino silicone” denotes any silicone not comprising any primary, secondary or tertiary amine groups or quaternary ammonium groups.

According to a preferred embodiment of the present invention, the compound(s) of formula (III) are such that:
R1 independently represents a hydroxyl group or an alkoxy group containing from 1 to 2 carbon atoms such as methoxy or ethoxy, or an alkyl group containing from 1 to 2 carbon atoms such as methyl;
R2 independently represents an alkyl group containing from 1 to 10 carbon atoms, preferably from 1 to 4 carbon atoms and more preferentially from 1 to 2 carbon atoms, such as a methyl;
R3 represents a hydroxyl group; an alkyl group containing from 1 to 10 carbon atoms, notably from 1 to 4 carbon atoms or an alkoxy group containing from 1 to 2 carbon atoms;
X represents a hydrogen atom or a hydrocarbon-based radical containing from 1 to 2 carbon atoms, and p denotes an integer ranging from 0 to 3;
m′ denotes an integer ranging from 1 to 3, n′ denotes an integer ranging from 0 to 2 and m′+n′ = 3;
m denotes an integer ranging from 1 to 3, n denotes an integer ranging from 0 to 2 and m+n = 3;
j is equal to 0;
k is equal to 0;
q denotes an integer equal to 0 or 1;
t denotes an integer equal to 0 or 1; and
y denotes an integer ranging from 0 to 10, z denotes an integer ranging from 0 to 500 with x+z ranging from 0 to 500 and x+y+z ≥ 4; and
it being understood that if X represents a hydrogen atom, then t = 0 and p = 1 and if p = 0, then t = 1 and q = 1 and that at least one of the radicals R₁ or R₃ denotes a hydroxyl radical or an alkoxy radical containing from 1 to 2 carbon atoms.

According to an even more preferred embodiment of the present invention, the compound(s) of formula (III) are such that:
R1 independently represents a hydroxyl group or an alkoxy group such as methoxy or ethoxy, or an alkyl group containing from 1 to 2 carbon atoms such as methyl;
R2 independently represents an alkyl group containing from 1 to 10 carbon atoms, preferably from 1 to 4 carbon atoms and more preferentially from 1 to 2 carbon atoms, such as a methyl;
m denotes an integer ranging from 1 to 3, n denotes an integer ranging from 0 to 2 and m+n = 3, and in particular m = 3;
j is equal to 0;
y is equal to 0;
z denotes an integer ranging from 0 to 500; and
x+z ≥ 4;
it being understood that at least one of the radicals R1 denotes a hydroxyl radical or an alkoxy radical containing from 1 to 2 carbon atoms.

The nonamino silicone(s) of formula (III) used in the context of the invention may be chosen from:
the compounds of formula (Illa) below:

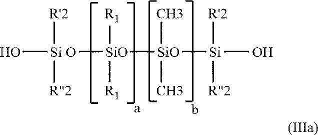

in which:
R₁, which may be identical or different, independently represents a hydroxyl group; an alkyl group containing from 1 to 10 carbon atoms, preferably 1 to 4 carbon atoms, notably 1 to 2 carbon atoms such as a methyl; an alkoxy group containing from 1 to 2 carbon atoms; or a group —(CH₂)ₛ—Si(R₄)₃ in which s denotes an integer ranging from 1 to 4 such as 2 and R₄ independently denotes an alkoxy radical containing from 1 to 2 carbon atoms;
R′₂ and R″₂ independently represent an alkyl group containing from 1 to 10 carbon atoms, preferably from 1 to 4 carbon atoms, notably 1 to 2 carbon atoms such as a methyl;
a denotes an integer ranging from 0 to 10, b denotes an integer ranging from 0 to 500 with a+b ≥ 4.
the compounds of formula (IIIb) below: [0066]

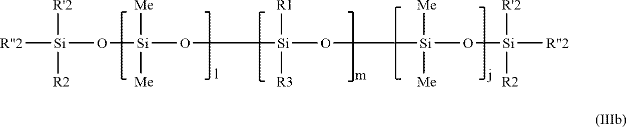

in which:
R1 independently represents an alkyl group containing from 1 to 10 carbon atoms, preferably from 1 to 4 carbon atoms and most particularly a methyl; or an alkoxy group containing from 1 to 2 carbon atoms;
R₂, R′₂ and R″₂ independently represent an alkyl group containing from 1 to 10 carbon atoms, preferably from 1 to 4 carbon atoms and in particular from 1 to 2 carbon atoms such as a methyl or an ethyl;
R₃ represents a group -Xp-Si(OR₂)₃ with X representing a saturated divalent hydrocarbon-based radical containing from 1 to 2 carbon atoms, and p denoting an integer ranging from 1 to 3;
m denotes an integer ranging from 0 to 3;
i denotes an integer ranging from 0 to 10, j denotes an integer ranging from 0 to 500 and i+j ranging from 0 to 510 with i+j ≥4;
and the compounds of formula (IIIc) below: [0069]

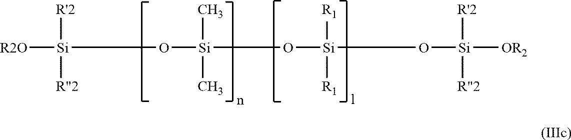

in which:
R₁, which may be identical or different, represents an alkyl group containing from 1 to 10 carbon atoms, preferably from 1 to 4 carbon atoms, more preferentially from 1 to 2 carbon atoms such as a methyl; an alkoxy group containing from 1 to 2 carbon atoms; or a group —O—(X)_{P}—Si(OR₂)₃ with X representing a saturated divalent hydrocarbon-based radical containing from 1 to 2 carbon atoms, and p denoting an integer ranging from 0 to 3;
R₂ independently represents an alkyl group containing from 1 to 10 carbon atoms, preferably from 1 to 4 carbon atoms and more preferentially from 1 to 2 carbon atoms such as a methyl or ethyl;
R′₂ and R″₂ independently represent an alkyl group containing from 1 to 10 carbon atoms, preferably from 1 to 4 carbon atoms, more preferentially from 1 to 2 carbon atoms such as a methyl or ethyl, or an alkoxy group containing from 1 to 10 carbon atoms, preferably from 1 to 4 carbon atoms, more preferentially from 1 to 2 carbon atoms such as a methoxy or ethoxy;
i denotes an integer ranging from 0 to 10, n denotes an integer ranging from 0 to 500 with n+i ranging from 0 to 510 with n+i ≥ 4;
and mixtures thereof.

Among the nonamino silicones of formula (llla), mention may be made of polydimethylsiloxanes (PDMS) bearing hydroxyl end functions, such as the compounds sold by the company Shin-Etsu under the name KF-9701 or X-21-5841, or those sold by the company Sigma-Aldrich under the reference 481939 (Mn ^{~}550, ^{~}25 cSt), 481955 (^{~}65 cSt), or 481963 (^{~}750 cSt).

Mention may also be made of the compounds sold by the company Gelest under the name DMS-S12 (16⁻32 cSt), DMS-S15 (45-85 cst), DMS-S21 (90-120 cst), DMS-S27 (700-800 cst) or DMS-S31 (^{~}1000 cSt).

Among the nonamino silicones of formula (lllb), mention may be made of polydimethylsiloxanes (PDMS) bearing trialkoxysilane side functions, such as those sold by the company Siltech under the name Silmer TMS C50.

Among the nonamino silicones of formula (lllc), mention may be made of polydimethylsiloxanes (PDMS) bearing trialkoxysilane end functions, such as those sold by the company Power Chemical under the name SiSiB® PF2110, or those sold by the company Siltech under the name Silmer TMS Di-10 or Silmer TMS Di-50.

According to a preferred embodiment, the nonamino silicone(s) used in the context of the invention are chosen from the compounds of formula (llla) in which:
R₁ independently represents an alkyl group containing from 1 to 10 carbon atoms, preferably from 1 to 4 carbon atoms and more particularly from 1 to 2 carbon atoms, such as a methyl;
R′₂ and R″₂ independently represent an alkyl group containing from 1 to 10 carbon atoms, preferably an alkyl group containing from 1 to 4 carbon atoms and more particularly from 1 to 2 carbon atoms such as a methyl;
b denotes an integer ranging from 0 to 10, a denotes an integer ranging from 0 to 5 with a+b ≥ 4.

According to another preferred embodiment, the nonamino silicone(s) used in the context of the invention are chosen from the compounds of formula (lllc) in which:
R₁ independently represents an alkyl group containing from 1 to 10 carbon atoms, preferably from 1 to 4 carbon atoms and notably a methyl;
R₂ represents an alkyl group containing from 1 to 10 carbon atoms, preferably from 1 to 4 carbon atoms and in particular from 1 to 2 carbon atoms, such as a methyl or an ethyl;
R′₂ and R″₂ independently represent an alkoxy group containing from 1 to 10 carbon atoms, preferably from 1 to 4 carbon atoms and more preferentially from 1 to 2 carbon atoms such as a methoxy or ethoxy;
i denotes an integer ranging from 0 to 10, n denotes an integer ranging from 0 to 500 and n+i ranging from 0 to 510 with n+i ≥ 4.

Preferably, the nonamino silicone(s) used in the context of the invention have a molecular mass of between 400 and 26000 g/mol.

Composition (F) according to the invention may comprise one or more nonamino silicones of formula (III) present in a total amount ranging from 0.1% to 30% by weight, preferably from 0.1% to 25% by weight relative to the total weight of composition (F).

### Mass Ratio

Composition (F) according to the invention comprises an alkoxysilane of formula (l) and alkoxysilane of formula (ll)/nonamino silicone of formula (III) mass ratio ranging from 95/5 to 5/95.

Preferably, the alkoxysilane of formula (l) and alkoxysilane of formula (ll)/nonamino silicone of formula (III) mass ratio ranges from 90/10 to 10/90, preferentially from 60/40 to 40/60.

According to a particular embodiment, the TEOS and octyltriethoxysilane/nonamino silicone mass ratio ranges from 90/10 to 10/90, preferably from 60/40 to 40/60.

### Coloring Agent

Composition (F) according to the invention optionally comprises at least one coloring agent chosen from pigments, direct dyes and mixtures thereof.

Preferably, composition (F) according to the invention comprises one or more pigments.

The term “pigment” refers to any pigment that gives color to keratin materials. Their solubility in water at 25° C. and at atmospheric pressure (760 mmHg) is less than 0.05% by weight, and preferably less than 0.01%.

The pigments that may be used are notably chosen from the organic and/or mineral pigments known in the art, notably those described in Kirk-Othmer’s Encyclopedia of Chemical Technology and in Ullmann’s Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann’s encyclopedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann’s Encyclopedia in the chapter on organic pigments.

The organic pigment may notably be chosen from nitroso, nitro, azo, xanthene, pyrene, quinoline, quinoline, anthraquinone, triphenylmethane, fluorane, phthalocyanine, metal-complex, isoindolinone, isoindoline, quinacridone, perinone, perylene, diketopyrrolopyrrole, indigo, thioindigo, dioxazine, triphenylmethane and quinophthalone compounds.

In particular, the white or colored organic pigments may be chosen from carmine, carbon black, aniline black, azo yellow, quinacridone, phthalocyanine blue, the blue pigments codified in the Color Index under the references Cl 42090, 69800, 69825, 74100, 74160, the yellow pigments codified in the Color Index under the references Cl 11680, 11710, 19140, 20040, 21100, 21108, 47000, 47005, the green pigments codified in the Color Index under the references Cl 61565, 61570, 74260, the orange pigments codified in the Color Index under the references Cl 11725, 45370, 71105, the red pigments codified in the Color Index under the references Cl 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow IOG: Yellow 3 pigment (Cl 11710);
Cosmenyl Yellow G: Yellow 1 pigment (Cl 11680);
Cosmenyl Orange GR: Orange 43 pigment (Cl 71105);
-Cosmenyl Red R: Red 4 pigment (Cl 12085);
Cosmenyl Carmine FB: Red 5 pigment (Cl 12490);
Cosmenyl Violet RL: Violet 23 pigment (Cl 51319);
Cosmenyl Blue A2R: Blue 15.1 pigment (Cl 74160);
Cosmenyl Green GG: Green 7 pigment (Cl 74260);
Cosmenyl Black R: Black 7 pigment (Cl 77266).

The pigments in accordance with the invention may also be in the form of composite pigments, as described in patent EP 1 184 426. These composite pigments may be composed notably of particles including a mineral core, at least one binder for attaching the organic pigments to the core, and at least one organic pigment which at least partially covers the core.

The organic pigment may also be a lake. The term “lake” refers to dyes adsorbed onto insoluble particles, the assembly thus obtained remaining insoluble during use.

The mineral substrates onto which the dyes are adsorbed are, for example, alumina, silica, calcium sodium borosilicate or calcium aluminum borosilicate and aluminum.

Among the dyes, mention may be made of carminic acid. Mention may also be made of the dyes known under the following names: D&C Red 21 (Cl 45 380), D&C Orange 5 (Cl 45 370), D&C Red 27 (Cl 45 410), D&C Orange 10 (Cl 45 425), D&C Red 3 (Cl 45 430), D&C Red 4 (Cl 15 510), D&C Red 33 (Cl 17 200), D&C Yellow 5 (Cl 19 140), D&C Yellow 6 (Cl 15 985), D&C Green (Cl 61 570), D&C Yellow 1 O (Cl 77 002), D&C Green 3 (Cl 42 053), D&C Blue 1 (Cl 42 090).

An example of a lake that may be mentioned is the product known under the following name: D&C Red 7 (Cl 15 850:1).

The pigment may also be a pigment with special effects. The term “pigments with special effects” means pigments that generally create a colored appearance (characterized by a certain shade, a certain vivacity and a certain level of luminance) that is non-uniform and that changes as a function of the conditions of observation (light, temperature, angles of observation, etc.). They thereby differ from colored pigments, which afford a standard uniform opaque, semitransparent or transparent shade.

Several types of pigments with special effects exist: those with a low refractive index, such as fluorescent or photochromic pigments, and those with a higher refractive index, such as nacres, interference pigments or glitter flakes.

Examples of pigments with special effects that may be mentioned include nacreous pigments such as mica coated with titanium or with bismuth oxychloride, colored nacreous pigments such as mica covered with titanium and with iron oxides, mica covered with iron oxide, mica covered with titanium and notably with ferric blue or with chromium oxide, mica covered with titanium and with an organic pigment as defined previously, and also nacreous pigments based on bismuth oxychloride. Nacreous pigments that may be mentioned include the Cellini nacres sold by BASF (mica-TiO2-lake), Prestige sold by Eckart (mica-TiO2), Prestige Bronze sold by Eckart (mica-Fe203), and Colorona sold by Merck (mica-TiO2-Fe2O3).

Mention may also be made of the gold-colored nacres sold notably by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold notably by the company Merck under the name Bronze fine (17384) (Colorona) and Bronze (17353) (Colorona) and by the company BASF under the name Super bronze (Cloisonne); the orange nacres sold notably by the company BASF under the name Orange 363C (Cloisonne) and Orange MCR 101 (Cosmica) and by the company Merck under the name Passion orange (Colorona) and Matte orange (17449) (Microna); the brown nacres sold notably by the company BASF under the name Nu-antique copper 340XB (Cloisonne) and Brown CL4509 (Chromalite); the nacres with a copper tint sold notably by the company BASF under the name Copper 340A (Timica); the nacres with a red tint sold notably by the company Merck under the name Sienna fine (17386) (Colorona); the nacres with a yellow tint sold notably by the company BASF under the name Yellow (4502) (Chromalite); the red nacres with a gold tint sold notably by the company BASF under the name Sunstone G012 (Gemtone); the pink nacres sold notably by the company BASF under the name Tan opale G005 (Gemtone); the black nacres with a gold tint sold notably by the company BASF under the name Nu antique bronze 240 AB (Timica), the blue nacres sold notably by the company Merck under the name Matte blue (17433) (Microna), the white nacres with a silvery tint sold notably by the company Merck under the name Xirona Silver, and the golden-green pink-orange nacres sold notably by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are notably sold under the name Metashine MC1080RY by the company Toyal.

Finally, examples of nacres that may also be mentioned include polyethylene terephthalate glitter flakes, notably those sold by the company Meadowbrook Inventions under the name Silver 1P 0.004X0.004 (silver glitter flakes). It is also possible to envisage multilayer pigments based on synthetic substrates, such as alumina, silica, calcium sodium borosilicate, calcium aluminum borosilicate and aluminum.

The pigments with special effects may also be chosen from reflective particles, i.e. notably from particles whose size, structure, notably the thickness of the layer(s) of which they are made and their physical and chemical nature, and surface state, allow them to reflect incident light. This reflection may, where appropriate, have an intensity sufficient to create at the surface of the composition or of the mixture, when it is applied to the support to be made up, highlight points that are visible to the naked eye, i.e. more luminous points that contrast with their environment making them appear to sparkle.

The reflective particles may be selected so as not to significantly alter the coloring effect generated by the coloring agents with which they are combined, and more particularly so as to optimize this effect in terms of color rendition. They may more particularly have a yellow, pink, red, bronze, orange, brown, gold and/or coppery color or tint.

These particles may have varied forms and may notably be in platelet or globular form, in particular in spherical form.

The reflective particles, whatever their form, may or may not have a multilayer structure and, in the case of a multilayer structure, may have, for example, at least one layer of uniform thickness, notably of a reflective material.

When the reflective particles do not have a multilayer structure, they may be composed, for example, of metal oxides, notably titanium or iron oxides obtained synthetically.

When the reflective particles have a multilayer structure, they may include, for example, a natural or synthetic substrate, notably a synthetic substrate at least partially coated with at least one layer of a reflective material, notably of at least one metal or metallic material. The substrate may be made of one or more organic and/or mineral materials.

More particularly, it may be chosen from glasses, ceramics, graphite, metal oxides, aluminas, silicas, silicates, notably aluminosilicates and borosilicates, and synthetic mica, and mixtures thereof, this list not being limiting.

The reflective material may include a layer of metal or of a metallic material.

Reflective particles are notably described in JP-A-09188830, JP-A-10158450, JP-A- 10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the names Crystal Star GF 550 and GF 2525 by this same company.

Use may also be made of particles comprising a metal substrate, such as silver, aluminum, iron, chromium, nickel, molybdenum, gold, copper, zinc, tin, magnesium, steel, bronze or titanium, said substrate being coated with at least one layer of at least one metal oxide, such as titanium oxide, aluminum oxide, iron oxide, cerium oxide, chromium oxide, silicon oxides and mixtures thereof.

Examples that may be mentioned include aluminum powder, bronze powder or copper powder coated with SiO2 sold under the name Visionaire by the company Eckart.

Mention may also be made of pigments with an interference effect which are not attached to a substrate, such as liquid crystals (Helicones HC from Wacker) or interference holographic glitter flakes (Geometric Pigments or Spectra fix from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colors, and also particular optical effects such as metallic effects or interference effects.

The size of the pigment used in the composition according to the present invention is generally between 10 nm and 200 µm, preferably between 20 nm and 80 µm and more preferentially between 30 nm and 50 µm.

The pigments may be dispersed in the composition by means of a dispersant.

The dispersant serves to protect the dispersed particles against agglomeration or flocculation thereof. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. In particular, esters of 12-hydroxystearic acid in particular and of Cs to C₂₀ fatty acid and of polyols such as glycerol or diglycerol are used, such as poly(12-hydroxystearic acid) stearate with a molecular weight of approximately 750 g/mol, such as the product sold under the name Solsperse 21000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent.

Thus, the pigments that have been surface-treated beforehand, which are useful in the context of the invention, are pigments that have totally or partially undergone a surface treatment of chemical, electronic, electrochemical, mechanochemical or mechanical nature, with an organic agent such as those described notably in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminum salts of fatty acids, for example aluminum stearate or laurate; metal alkoxides; polyethylene; (meth)acrylic polymers, for example polymethyl methacrylates; polymers and copolymers containing acrylate units; alkanolamines; silicone compounds, for example silicones, notably polydimethylsiloxanes; organofluorine compounds, for example perfluoroalkyl ethers; fluorosilicone compounds.

The surface-treated pigments that are useful in the composition may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent US 4 578 266.

An organic agent covalently bonded to the pigments will preferably be used.

The agent for the surface treatment may represent from 0.1% to 50% by weight relative to the total weight of the surface-treated pigment, preferably from 0.5% to 30% by weight and even more preferentially from 1% to 20% by weight relative to the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a magnesium myristate treatment, for instance the MM surface treatment sold by LCW;
an aluminum dimyristate treatment, such as the MI surface treatment sold by Miyoshi;
a perfluoropolymethylisopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF + ITT surface treatment sold by Daito.

According to a particular embodiment of the invention, the dispersant is present with organic or mineral pigments in submicron-sized particulate form in the dye composition.

The term “submicron” or “submicronic” refers to pigments having a particle size that has been micronized by a micronization method and having a mean particle size of less than a micrometer (µm), in particular between 0.1 and 0.9 µm, and preferably between 0.2 and 0.6 µm.

According to one embodiment, the dispersant and the pigment(s) are present in an amount (dispersant/pigment) of between ¼ and 4/1, particularly between 1.5/3.5 and 3.5/1 or better still between 1.75/3 and 3/1.

The dispersant(s) may therefore have a silicone backbone, such as silicone polyether and dispersants of aminosilicone type other than the alkoxysilanes described previously. Among the suitable dispersants, mention may be made of:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones with carboxyl groups such as X- 22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of aminosilicone type other than the alkoxysilanes described previously and are cationic.

Preferably, the pigment(s) are chosen from mineral, mixed mineral-organic or organic pigments.

In one variant of the invention, the pigment(s) according to the invention are organic pigments, preferentially organic pigments surface-treated with an organic agent chosen from silicone compounds. In another variant of the invention, the pigment(s) according to the invention are mineral pigments.

Composition (F) may comprise one or more direct dyes.

The term “direct dye” means natural and/or synthetic dyes, other than oxidation dyes. These are dyes that will spread superficially on the fiber.

They may be ionic or nonionic, preferably cationic or nonionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (IV) and (V) and the azo cationic dyes (VI) and (VII) below:

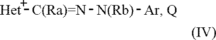

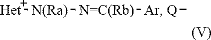

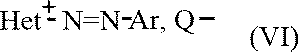

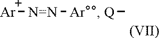

in which formulae (IV) to (VII):
Het⁺ represents a cationic heteroaryl radical, preferentially bearing an endocyclic cationic charge, such as imidazolium, indolium or pyridinium, which is optionally substituted, preferentially with at least one (C₁-C₈) alkyl group such as methyl;
Ar⁺ represents an aryl radical, such as phenyl or naphthyl, bearing an exocyclic cationic charge, preferentially ammonium, particularly tri(C₁-C₈)alkylammonium, such as trimethylammonium;
Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N-(C₁-C₈)alkyl-N-aryl(C₁-C₈)alkylamino or alternatively Ar represents a julolidine group;
Ar” represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
Ra and Rb, which may be identical or different, represent a hydrogen atom or a (C₁-C₈)alkyl group, which is optionally substituted, preferentially with a hydroxyl group;
or else the substituent Ra with a substituent of Het+ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
Q- represents an organic or mineral anionic counterion, such as a halide or an alkyl sulfate.

In particular, mention may be made of the azo and hydrazono direct dyes bearing an endocyclic cationic charge of formulae (IV) to (VII) as defined previously, more particularly, the cationic direct dyes bearing an endocyclic cationic charge described in patent applications WO 95/15144, WO 95/01772 and EP 714 954, preferentially the following direct dyes:

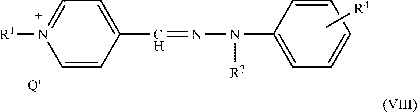

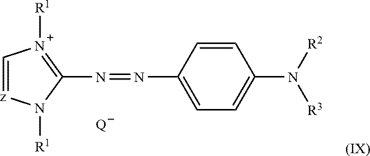

in which formulae (VIII) and (IX):
R¹ represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH,
Q- is an anionic counterion as defined previously, in particular a halide, such as chloride, or an alkyl sulfate, such as methyl sulfate or mesityl.

In particular, the dyes of formulae (VIII) and (IX) are chosen from Basic Red 51, Basic Yellow 87 and Basic Orange 31 or derivatives thereof with Q′ being an anionic counterion as defined previously, particularly halide such as chloride, or an alkyl sulfate such as methyl sulfate or mesityl.

The direct dyes may be chosen from anionic direct dyes. The anionic direct dyes of the invention are dyes commonly referred to as “acid” direct dyes owing to their affinity for alkaline substances. The term “anionic direct dye” means any direct dye including in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

As acid dyes according to the invention, mention may be made of the dyes of formulae (X), (X′), (XI), (XI′), (XII), (XII′), (XIII), (XIII′), (XIV), (XV), (XVI) and (XVII) below:

a) the diaryl anionic azo dyes of formula (X) or (X′):

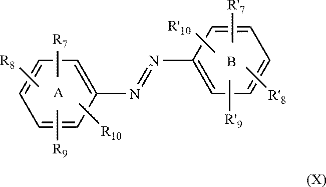

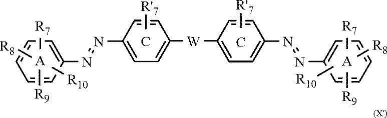

in which formulae (X) and (X′):
R₇, R₈, R₉, R₁₀, R′₇, R’s, R′₉ and R′₁₀, which may be identical or different, represent a hydrogen atom or a group chosen from:
alkyl;
alkoxy, alkylthio;
hydroxyl, mercapto;
nitro, nitroso;
R°—C(X)—X′—, R°—X′—C(X)—, R°—X′—C(X)—X″— with R° representing a hydrogen atom or an alkyl or aryl group; X, X′ and X″, which may be identical or different, representing an oxygen or sulfur atom, or NR with R representing a hydrogen atom or an alkyl group;
(O)₂S(O—)—, M+ with M+ representing a hydrogen atom or a cationic counterion;
(O)CO^{—}—, M⁺with M⁺as defined previously;
R ”—S(O)₂—, with R″ representing a hydrogen atom or an alkyl, aryl, (di)(alkyl)amino or aryl(alkyl)amino group; preferentially a phenylamino or phenyl group;
R”’—S(O)₂—X′— with R‴ representing an optionally substituted alkyl or aryl group, X′ as defined previously;
(di)(alkyl)amino;
aryl(alkyl)amino optionally substituted with one or more groups chosen from i) nitro; ii) nitroso; iii) (O)2S(O-)-, M+ and iv) alkoxy with M+ as defined previously;
optionally substituted heteroaryl; preferentially a benzothiazolyl group;
cycloalkyl; notably cyclohexyl;
Ar—N═N— with Ar representing an optionally substituted aryl group, preferentially a phenyl optionally substituted with one or more alkyl, (O)2S(O—)—, M+ or phenylamino groups;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O—)—, M+; iv) hydroxyl; v) mercapto; vi) (di)(alkyl)amino; vii) R°—C(X)—X′—; viii) R°—X′—C(X)—; ix) R°—X′—C(X)—X″—; x) Ar—N═N— and xi) optionally substituted aryl(alkyl)amino; with M+, R°, X, X′, X″ and Ar as defined previously;
W represents a sigma bond σ, an oxygen or sulfur atom, or a divalent radical i) —NR— with R as defined previously, or ii) methylene —C(Ra)(Rb)— with Ra and Rb, which may be identical or different, representing a hydrogen atom or an aryl group, or alternatively Ra and Rb form, with the carbon atom that bears them, a spiro cycloalkyl; preferentially, W represents a sulfur atom or Ra and Rb together form a cyclohexyl;
it being understood that formulae (X) and (X′) comprise at least one sulfonate radical (O)₂S(O—)—, M+ or one carboxylate radical (O)CO--, M+ on one of the rings A, A′, B, B′ or C; preferentially sodium sulfonate;

As examples of dyes of formula (X), mention may be made of: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food Yellow 3 or Sunset Yellow;

and as examples of dyes of formula (X′), mention may be made of: Acid Red 111, Acid Red 134, Acid Yellow 38.

b) the pyrazolone anionic azo dyes of formulae (XI) and (XI′):

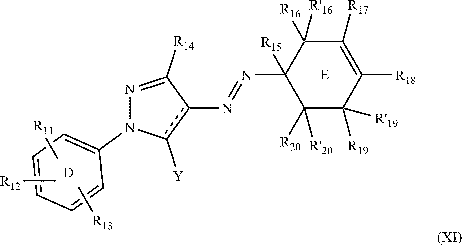

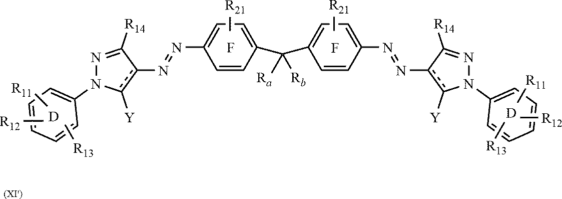

in which formulae (XI) and (XI′)—
R₁₁, R₁₂ and R₁₃, which may be identical or different, represent a hydrogen or halogen atom, an alkyl group or —(O)₂S(O—), M+ with M+ as defined previously;
R₁₄ represents a hydrogen atom, an alkyl group or a group —C(O)O—, M+ with M+ as defined previously;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom, or a group chosen from:
(O)₂S(O—)—, M+ with M+ as defined previously;
Ar—O—S(O)₂— with Ar representing an optionally substituted aryl group; preferentially a phenyl optionally substituted with one or more alkyl groups;
R₁₉ and R₂₀ together form either a double bond, or a benzo group D′, which is optionally su bstituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Rₐ and R_{b}, which may be identical or different, are as defined previously; preferentially, Rₐ represents a hydrogen atom and R_{b} represents an aryl group;
Y represents either a hydroxyl group or an oxo group;

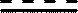

represents a single bond when Y is an oxo group; and represents a double bond when Y represents a hydroxyl group;
it being understood that formulae (XI) and (XI′) comprise at least one sulfonate radical (O)₂S(O—)—, M+ or one carboxylate radical —C(O)O—, M+ on one of the rings D or E; preferentially sodium sulfonate;

As examples of dyes of formula (XI), mention may be made of: Acid Red 195, Acid Yellow 23, Acid Yellow 27, Acid Yellow 76, and as examples of dyes of formula (XI′), mention may be made of: Acid Yellow 17;

c) the anthraquinone dyes of formulae (XII) and (XII′):

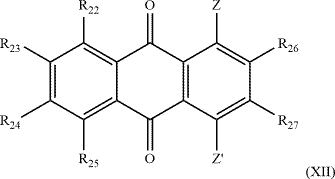

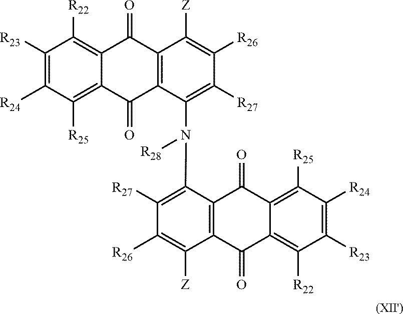

in which formulae (XII) and (XII′):
R₂₂, R₂₃, R₂₄, R₂₅, R₂₆ and R₂₇, which may be identical or different, represent a hydrogen or halogen atom, or a group chosen from:
alkyl;
hydroxyl, mercapto;
alkoxy, alkylthio;
optionally substituted aryloxy or arylthio, preferentially substituted with one or more groups chosen from alkyl and (O)₂S(O—)—, M+ with M+ as defined previously;
aryl(alkyl)amino optionally substituted with one or more groups chosen from alkyl and (O)2S(O—)—, M+ with M+ as defined previously;
(di)(alkyl)amino;
(di)(hydroxyalkyl)amino;
— (O)₂S(O—)—, M+ with M+ as defined previously;

Z′ represents a hydrogen atom or a group NR28R29 with R28 and R29, which may be identical or different, representing a hydrogen atom or a group chosen from:
alkyl;
polyhydroxyalkyl such as hydroxyethyl;
aryl optionally substituted with one or more groups, particularly i) alkyl such as methyl, n-dodecyl, n-butyl; ii) (O)₂S(O—)—, M+ with M+ as defined previously; iii) R°—C(X)—X′—, R°—X′—C(X)— , R°—X′—C(X)—X″— with R°, X, X′ and X″ as defined previously, preferentially R° represents an alkyl group;
cycloalkyl; notably cyclohexyl;

Z, represents a group chosen from hydroxyl and NR′28R′29 with R′28 and R′29, which may be identical or different, representing the same atoms or groups as R28 and R29 as defined previously; it being understood that formulae (XII) and (XII′) comprise at least one sulfonate radical (O)₂S(O—)—, M+ or one carboxylate radical C(O)O—, M+; preferentially sodium sulfonate;

As examples of dyes of formula (XII), mention may be made of: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT violet No. 2; and, as an example of a dye of formula (XII′), mention may be made of: Acid Black 48;

d) the nitro dyes of formulae (XIII) and (XIII′):

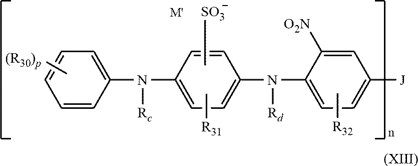

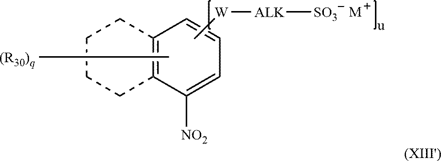

in which formulae (XIII) and (XIII′):
R₃₀, R₃₁ and R₃₂, which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
polyhaloalkyl;
R°—C(X)—X′—, R°—X′—C(X)—, R°—X′—C(X)—X″— with R°, X, X′ and X″ as defined previously;
— (O)₂S(O—)—, M+ with M+ as defined previously;
(O)CO⁻-, M⁺ with M′ as defined previously;
(di)(alkyl)amino;
(di)(hydroxyalkyl)amino;
heterocycloalkyl such as piperidino, piperazino or morpholino; in particular, R₃₀, R₃₁ and R₃₂ represent a hydrogen atom;
Rc and Rd, which may be identical or different, represent a hydrogen atom or an alkyl group;
W is as defined previously; W particularly represents an —NH— group;
ALK represents a linear or branched divalent C₁-C₆ alkylene group; in particular, ALK represents a —CH₂—CH₂— group;
n is 1 or 2;
p represents an integer inclusively between 1 and 5;
q represents an integer inclusively between 1 and 4;
u is 0 or 1;
when n is 1, J represents a nitro or nitroso group; particularly nitro;
when n is 2, J represents an oxygen or sulfur atom, or a divalent radical —S(O)m— with m representing an integer 1 or 2; preferentially, J represents an —SO2— radical;
M′ represents a hydrogen atom or a cationic counterion;

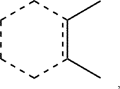

which may be present or absent, represents a benzo group optionally substituted with one or more groups R30 as defined previously;
it being understood that formulae (XIII) and (XIII′) comprise at least one sulfonate radical (O)₂S(O—)—, M+ or one carboxylate radical —C(O)O—, M+; preferentially sodium sulfonate.

As examples of dyes of formula (XIII), mention may be made of: Acid Brown 13 and Acid Orange 3; as examples of dyes of formula (XIII′), mention may be made of: Acid Yellow 1, the sodium salt of 2,4-dinitro-1-naphthol-7-sulfonic acid, 2-piperidino-5-nitrobenzenesulfonic acid, 2-(4′-N,N-(2″-hydroxyethyl)amino-2′-nitro)anilineethanesulfonic acid, 4-β-hydroxyethylamino-3-nitrobenzenesulfonic acid; EXT D&C Yellow 7;

e) the triarylmethane dyes of formula (XIV):

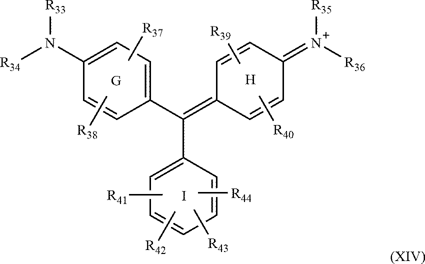

in which formula (XIV):
R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O—)—, M+ with M+ and m as defined previously;
R₃₇, R₃₈, R₃₉, R₄₀, R₄₁, R₄₂, R₄₃ and R₄₄, which may be identical or different, represent a hydrogen atom or a group chosen from:
alkyl;
alkoxy, alkylthio;
(di)(alkyl)amino;
hydroxyl, mercapto;
nitro, nitroso;
R°—C(X)—X′—, R°—X′—C(X)—, R°—X′—C(X)—X″— with R° representing a hydrogen atom or an alkyl or aryl group; X, X′ and X″, which may be identical or different, representing an oxygen or sulfur atom, or NR with R representing a hydrogen atom or an alkyl group;
(O)₂S(O—)—, M+ with M+ representing a hydrogen atom or a cationic counterion;
(O)CO^{—}—, M⁺ with M⁺ as defined previously;
or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O—)—, M+; iv) hydroxyl; v) mercapto; vi) (di)(alkyl)amino; vii) R°—C(X)—X′—; viii) R°—X′—C(X)— and ix) R°—X′—C(X)—X″—; with M+, R°, X, X′ and X″ as defined previously;
in particular, R₃₇ to R₄₀ represent a hydrogen atom, and R₄₁ to R₄₄, which may be identical or different, represent a hydroxyl group or (O)₂S(O—)—, M+; and when R₄₃ with R₄₄ together form a benzo group, it is preferentially substituted with an (O)₂S(O—)— group;
it being understood that at least one of the rings G, H, I or I′ comprises at least one sulfonate radical (O)₂S(O—)— or a carboxylate radical —C(O)O—; preferentially sulfonate;

As examples of dyes of formula (XIV), mention may be made of: Acid Blue 1; Acid Blue 3; Acid Blue 7, Acid Blue 9; Acid Violet 49; Acid Green 3; Acid Green 5 and Acid Green 50;

e) the xanthene-based dyes of formula (XV):

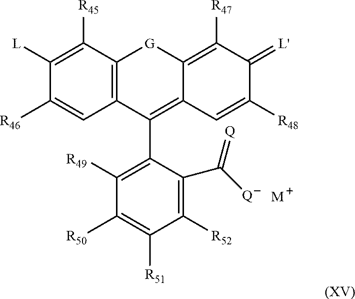

in which formula (XV):
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
alkyl;
alkoxy, alkylthio;
hydroxyl, mercapto;
nitro, nitroso;
(O)₂S(O—)—, M+ with M+ representing a hydrogen atom or a cationic counterion;
(O)CO⁻-, M⁺ with M⁺ as defined previously;
particularly R₅₃, R₅₄, R₅₅ and R₄₈ represent a hydrogen or halogen atom;
G represents an oxygen or sulfur atom or a group NRe with Re as defined previously; particularly G represents an oxygen atom;
L represents an alkoxide O—, M+; a thioalkoxide S—, M+ or a group NRf, with Rf representing a hydrogen atom or an alkyl group, and M+ as defined previously; M+ is particularly sodium or potassium;
L′ represents an oxygen or sulfur atom or an ammonium group: N+RfRg, with Rf and Rg, which may be identical or different, representing a hydrogen atom or an optionally substituted alkyl or aryl group; L′ particularly represents an oxygen atom or a phenylamino group optionally substituted with one or more alkyl or (O)ₘS(O—)—, M+ groups with m and M+ as defined previously;
Q and Q′, which may be identical or different, represent an oxygen or sulfur atom; particularly, Q and Q′ represent an oxygen atom;
M+ is as defined previously;

As examples of dyes of formula (XV), mention may be made of: Acid Yellow 73; Acid Red 51; Acid Red 52; Acid Red 87; Acid Red 92; Acid Red 95; Acid Violet 9;

f) the indole-based dyes of formula (XVI):

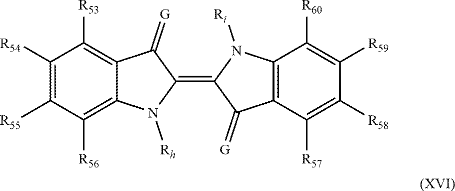

R₅₃, R₅₄, R₅₅, R₅₆, R₅₇, R₅₈, R₅₉ and R₆₀, which may be identical or different, represent a hydrogen atom or a group chosen from:
-alkyl;
alkoxy, alkylthio;
hydroxyl, mercapto;
nitro, nitroso;
R°—C(X)—X′—, R°—X′—C(X)—, R°—X′—C(X)—X″— with R° representing a hydrogen atom or an alkyl or aryl group; X, X′ and X″, which may be identical or different, representing an oxygen or sulfur atom, or NR with R representing a hydrogen atom or an alkyl group;
(O)₂S(O—)—, M+ with M+ representing a hydrogen atom or a cationic counterion;
(O)CO⁻-, M⁺ with M⁺ as defined previously;
G represents an oxygen or sulfur atom or a group NRe with Re as defined previously; particularly, G represents an oxygen atom;
Ri and Rh, which may be identical or different, represent a hydrogen atom or an alkyl group;
it being understood that formula (XVI) comprises at least one sulfonate radical (O)₂S(O—)—, M+ or one carboxylate radical —C(O)O—, M+; preferentially sodium sulfonate.

As examples of dyes of formula (XVI), mention may be made of: Acid Blue 74.

g) the quinoline-based dyes of formula (XVII):

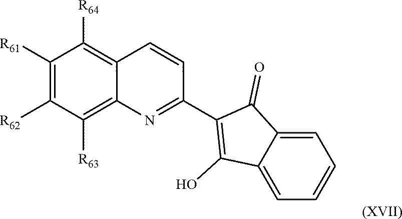

R₆₁ represents a hydrogen or halogen atom or an alkyl group;
R₆₂, R₆₃ and R₆₄, which may be identical or different, represent a hydrogen atom or a group (O)₂S(O—)—, M+ with M+ representing a hydrogen atom or a cationic counterion;
or alternatively R₆₁ with R₆₂, or R₆₁ with R₆₄, together form a benzo group optionally substituted with one or more groups (O)₂S(O—)—, M+ with M+ representing a hydrogen atom or a cationic counterion;
it being understood that formula (XVII) comprises at least one sulfonate radical (O)₂S(O—)—, M+, preferentially sodium sulfonate.

As examples of dyes of formula (XVII), mention may be made of: Acid Yellow 2, Acid Yellow 3 and Acid Yellow 5.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

Preferably, the direct dye(s) are chosen from anionic direct dyes.

The pigments may be present in concentrations ranging from 0.05% to 15% by weight and preferably from 0.1% to 10% by weight relative to the total weight of the composition.

The direct dye(s) may be present in concentrations ranging from 0.001% to 10% by weight relative to the total weight of the composition, preferably from 0.005% to 5% by weight relative to the total weight of the composition.

Composition (F) according to the invention may comprise water. Preferably, water is present in a content ranging from 0.1% to 20% by weight, more preferentially from 0.5% to 15% by weight relative to the total weight of the composition.

Composition (F) according to the invention may comprise less than 10% by weight of water relative to the total weight of composition (F).

### Solvents

Composition (F) according to the invention may comprise one or more organic solvents.

Examples of organic solvents that may be mentioned include lower C₁-C₄ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

Preferably, composition (F) comprises one or more organic solvents chosen from C₁-C₄ lower alkanols, more preferentially ethanol.

The organic solvents may be present in a total amount inclusively between 1% and 80% by weight approximately relative to the total weight of composition (F), preferably between 1% and 70% by weight and more preferentially inclusively between 3% and 70% by weight relative to the total weight of composition (F).

### Additives

Composition (F) may also contain any adjuvant or additive usually used in the field of hair treatments.

Among the additives that may be contained in the composition, mention may be made of reducing agents, thickeners, softeners, antifoams, moisturizers, solubilizers, fragrances, anionic, cationic, nonionic or amphoteric surfactants, proteins, vitamins, polymers other than the compounds of the invention, preserving agents, oils, waxes and mixtures thereof.

Composition (F) according to the invention may notably be in the form of a suspension, a dispersion, a gel, an emulsion, notably an oil-in-water (O/W) or water-in-oil (W/O) emulsion, or a multiple emulsion (W/O/W or polyol/O/W or O/W/O), in the form of a cream, a mousse, a stick, a dispersion of vesicles, notably of ionic or nonionic lipids, or a two-phase or multi-phase lotion.

In a preferred embodiment, the cosmetic composition according to the invention is an aqueous-alcoholic system.

A person skilled in the art may select the appropriate presentation form, and also the method for preparing it, on the basis of his general knowledge, taking into account firstly the nature of the constituents used, notably their solubility in the support, and secondly the intended application of the composition.

The present invention also relates to the use of composition (F) as described above for the cosmetic treatment of, in particular for dyeing, keratin fibers, in particular human keratin fibers such as the hair.

According to a preferred embodiment of the invention, composition (F) comprises tetraethoxysilane (TEOS), n-octyltriethoxysilane and at least one nonamino silicone chosen from the compounds of formula (IIIa) as described previously.

According to another preferred embodiment of the invention, composition (F) comprises tetraethoxysilane (TEOS), n-octyltriethoxysilane and at least one nonamino silicone chosen from the compounds of formula (IIIc) as described previously.

### Process for Treating Keratin Fibers

The present invention also relates to a process for treating human keratin fibers such as the hair, which consists in extemporaneously mixing, at the time of use, two compositions (A) and (B) and in applying said mixture, composition (F), to the fibers, with:
composition (A) comprising at least one alkoxysilane chosen from the compounds of formula (I), oligomers thereof and/or mixtures thereof as described previously and at least one alkoxysilane chosen from the compounds of formula (II), oligomers thereof and/or mixtures thereof as described previously, and optionally water;
composition (B) comprising at least one nonamino silicone of formula (III) as described previously;
it being understood that composition (A) and/or composition (B) optionally comprise at least one coloring agent chosen from pigments, direct dyes and mixtures thereof.

Preferably, the treatment process comprises water present in composition (A) and/or present on the keratin fibers moistened prior to the application.

Preferably, the process for treating human keratin fibers is a process for dyeing human keratin fibers such as the hair.

Preferably, composition (A) does not comprise any nonamino silicone of formula (III) and composition (B) does not comprise any alkoxysilane of formula (I), oligomers thereof and/or mixtures thereof, or any alkoxysilane of formula (II), oligomers thereof and/or mixtures thereof.

Preferably, composition (A) comprises water.

More preferentially, composition (A) and/or composition (B) comprise one or more organic solvents, better still chosen from C₁-C₄ lower alkanols, more preferentially ethanol.

A coloring agent chosen from pigments, direct dyes and mixtures thereof may be present in composition (A). Thus, composition (A) and composition (B) are mixed extemporaneously at the time of use before being applied to the keratin fibers.

A coloring agent chosen from pigments, direct dyes and mixtures thereof may be present in composition (B). Thus, composition (A) and composition (B) are mixed extemporaneously at the time of use before being applied to the keratin fibers.

A coloring agent chosen from pigments, direct dyes and mixtures thereof may be alternatively present in a composition (C) different from compositions (A) and (B) and not containing any alkoxysilane of formula (I) or any alkoxysilane of formula (II) or any nonamino silicone of formula (III). Composition (A), composition (B) and composition (C) may be mixed extemporaneously at the time of use before being applied to the keratin fibers.

Preferably, composition A has an acidic pH. Preferably, the pH of composition A is between 1.5 and 6, preferably between 2 and 5 and more particularly between 2.5 and 4.5.

Preferably, the application of composition (F) to the keratin fibers is followed by a heating step, preferably at a temperature of greater than or equal to 30° C.

This heating step may be continuous or fractionated, for instance a heating step at a temperature of greater than or equal to 30° C., for example using a hairdryer or a heating lamp, followed by a heating step at a higher temperature, for example using a straightening iron. When the heating step is fractionated, a step at room temperature may be performed between two periods of heating of the keratin fibers.

According to a particular embodiment, the process of the invention is a process for treating human keratin fibers such as the hair, which consists in extemporaneously mixing, at the time of use, two compositions (A) and (B) and in applying the mixture to the fibers moistened with water, with:
composition (A) comprising at least one alkoxysilane of formula (I), oligomers thereof and/or mixtures thereof as described previously and at least one alkoxysilane of formula (II), oligomers thereof and/or mixtures thereof as described previously;
composition (B) comprising at least one nonamino silicone of formula (III) as described previously;
it being understood that composition (A) and/or composition (B) optionally comprise at least one coloring agent chosen from pigments, direct dyes and mixtures thereof.

Preferably, the mass ratio of alkoxysilane of formula (I) and alkoxysilane of formula (II)/nonamino silicone ranges from 95/5 to 5/95, preferentially from 90/10 to 10/90, more preferentially from 60/40 to 40/60.

According to a preferred embodiment of the invention, the process for treating human keratin fibers such as the hair, according to the invention, consists in extemporaneously mixing, at the time of use, two compositions (A) and (B) and in applying the mixture to the fibers, with:
composition (A) comprising at least one alkoxysilane chosen from the compounds of formula (I), oligomers thereof and/or mixtures thereof as described previously and at least one alkoxysilane chosen from the compounds of formula (II), oligomers thereof and/or mixtures thereof as described previously, and water, said composition (A) preferably being at acidic pH, notably between 2.5 and 4.5;
composition (B) comprising at least one nonamino silicone of formula (III) as described previously;
said application being followed by a heating step, preferably at a temperature of greater than or equal to 30° C., it being understood that composition (A) and/or composition (B) optionally comprise at least one coloring agent chosen from pigments, direct dyes and mixtures thereof.

Preferably, the process for treating human keratin fibers such as the hair, according to the invention, involves a step of treating the keratin fibers which consists in applying to said fibers a cosmetic composition (G) comprising at least one alkoxysilane chosen from the compounds of formula (XVIII), oligomers thereof and/or mixtures thereof as described below; said treatment step taking place before the application of composition (F) to the keratin fibers.

Preferably, the process for treating human keratin fibers such as the hair, comprises:
i) the application to said fibers of a composition (G) comprising at least one alkoxysilane of formula (XVIII),
ii) an optional leave-on time of from 1 to 30 minutes, notably from 1 to 20 minutes, in particular from 1 to 10 minutes, of composition (G) on said fibers,
iii) optional rinsing of the fibers,
iv) the application of a composition (F) as described previously, said composition (F) resulting from the extemporaneous mixing of compositions (A) and (B) described previously, and
vi) an optional heating step.

Preferably, a washing, rinsing, drying and/or draining step is performed after applying composition (G) to the keratin fibers and before applying composition (F) to the keratin fibers.

### Alkoxysilane of Formula (XVIII)

Composition (G) according to the invention comprises at least one alkoxysilane chosen from the compounds of formula (XVIII) below, oligomers thereof and/or mixtures thereof:

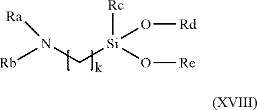

in which:
Ra and Rb, which may be identical or different, represent a hydrogen atom; an alkyl group containing from 1 to 20 carbon atoms, preferably from 1 to 10 carbon atoms and notably from 1 to 4 carbon atoms; a cycloalkyl group containing from 3 to 20 carbon atoms, notably 5 or 6 carbon atoms; an aryl group containing from 6 to 12 carbon atoms; an aminoalkyl group containing from 1 to 20 carbon atoms;
Rc represents a hydrogen atom; an alkyl group containing from 1 to 20 carbon atoms, preferably from 1 to 10 carbon atoms, more preferentially from 1 to 4 carbon atoms and in particular from 1 to 2 carbon atoms such as a methyl; an alkoxy group containing from 1 to 10 carbon atoms, preferably from 1 to 4 carbon atoms and in particular from 1 to 2 carbon atoms such as an ethoxy;
Rd and Re, which may be identical or different, represent an alkyl group containing from 1 to 10 carbon atoms, preferably from 1 to 4 carbon atoms, in particular from 1 to 2 carbon atoms, such as an ethyl;
k denotes an integer ranging from 0 to 5, in particular from 1 to 3.

Preferably, the compounds of formula (XVIII) are such that Ra and Rb, which are identical, represent a hydrogen atom or Ra denotes a hydrogen atom and Rb denotes a C₅-C₆ cycloalkyl radical such as cyclohexyl;
Rc represents an alkyl group containing from 1 to 10 carbon atoms, notably from 1 to 4 carbon atoms and in particular from 1 to 2 carbon atoms, preferably a methyl, or an alkoxy group containing from 1 to 4 carbon atoms, preferably from 1 to 2 carbon atoms, preferably an ethoxy;
Rd and Re, which may be identical or different, represent an alkyl group containing from 1 to 10 carbon atoms, preferably from 1 to 4 carbon atoms, such as an ethyl;
k denotes an integer ranging from 1 to 3 and more particularly 1 or 3.

More preferentially, the compounds of formula (XVIII) are such that Ra and Rb represent a hydrogen atom, Rc represents an ethoxy group, Rd and Re are identical and represent an ethyl and k is equal to 3.

According to another preferred form of the invention, the compounds of formula (XVIII) are such that Ra denotes a hydrogen atom, Rb represents a cyclohexyl radical, Rc represents an ethoxy group, Rd and Re are identical and represent an ethyl and k is equal to 1.

Among the alkoxysilanes of formula (XVIII), oligomers thereof and/or mixtures thereof, mention may notably be made of 3-aminopropyltriethoxysilane (APTES), 3-aminopropylmethyldiethoxysilane (APMDES) and N-cyclohexylaminomethyltriethoxysilane.

APTES may be purchased, for example, from the company Dow Corning under the name Xiameter OFS-6011 Silane or from the company Momentive Performance Materials under the name Silsoft A-1100.

The compounds of formula (XVIII) may also denote Dynasylan SIVO 210 or Dynasylan 1505 sold by the company Evonik.

The N-cycloheylaminomethyltriethoxysilane may be purchased, for example, from the company Wacker under the name Geniosil XL 926.

Preferably, the alkoxysilane(s) of formula (XVIII) are chosen from 3-aminopropyltriethoxysilane (APTES), 3-aminopropylmethyldiethoxysilane (APMDES), N-cyclohexylaminomethyltriethoxysilane and mixtures thereof, more preferentially 3-aminopropyltriethoxysilane (APTES).

Composition (G) according to the invention may comprise one or more alkoxysilanes of formula (XVIII), oligomers thereof and/or mixtures thereof present in a total amount ranging from 0.1% to 30% by weight, preferably from 0.5% to 25% by weight and better still from 0.5% to 20% by weight, relative to the total weight of the composition.

Preferably, composition (G) according to the invention comprises water. Preferably, water is present in a content ranging from 0.1% to 95% by weight, more preferentially from 0.5% to 90% by weight relative to the total weight of the composition.

Preferably, composition (G) comprises one or more organic or mineral acids, preferably mineral acids such as hydrochloric acid.

### Protocol

Composition (F) and/or composition (G) described above may be used on wet or dry keratin fibers, and also on any type of fair or dark, natural or dyed, permanent-waved, bleached or relaxed fibers.

According to a particular embodiment of the invention, the fibers are washed before applying composition (F) and/or composition (G).

The application to the fibers may be performed via any standard means, in particular using a comb, a fine brush, a coarse brush, a sponge or with the fingers.

Preferably, after applying composition (F) to the keratin fibers, there is a waiting time of between 1 minute and 14 days, in particular between 1 minute and 10 days, more particularly between 1 minute and 5 days.

Preferably, after applying the cosmetic composition (F) and/or composition (G) to the keratin fibers, the fibers are left to dry or are dried, for example at a temperature of greater than or equal to 30° C.

According to a particular embodiment, the fibers may be dried at a temperature of greater than or equal to 40° C. According to a particular embodiment, the fibers may be dried at a temperature of greater than 40° C. and less than or equal to 120° C.

Preferably, if the fibers are dried, they are dried, in addition to a supply of heat, with a flow of air.

During drying, a mechanical action may be exerted on the locks, such as combing, brushing or running the fingers through. This operation may similarly be performed once the fibers have been dried, naturally or otherwise.

The drying step may be performed with a drying device such as a hood, a hairdryer, a climazone, etc.

When the drying step is performed with a hood or a hairdryer, the drying temperature is between 40 and 110° C. and preferably between 50 and 90° C.

After the drying step, a shaping step may be performed, for example with a straightening iron; the temperature for the shaping step is between 110 and 220° C., preferably between 140 and 200° C.

Once the drying is complete, final rinsing or shampooing may optionally be performed.

According to a particular variant of the invention, a step of acidic rinsing at a pH of between 1 and 5 may be performed after the step of drying the locks and before the final shampoo wash.

According to a particular variant of the invention, a care of mask type with a pH of between 1 and 5 may be applied after the step of drying the locks and before the final shampoo wash, with a leave-on time of between 1 and 45 minutes, in particular between 5 minutes and 30 minutes and preferably about 15 minutes.

Preferably, the process of the invention is a process for dyeing keratin fibers, in particular human keratin fibers, preferably the hair, comprising the application to said fibers of a composition comprising:
a) at least one alkoxysilane of formula (I), oligomers thereof and/or mixtures thereof as described previously,
b) at least one alkoxysilane of formula (II), oligomers thereof and/or mixtures thereof as described previously,
c) at least one nonamino silicone of formula (III) as described previously,
d) at least one coloring agent chosen from pigments, direct dyes and mixtures thereof, and
e) optionally water.

The dyeing process may be repeated several times with identical or different compositions as described above, it being understood that each new application of composition is performed after a leave-on time ranging from 1 minute to several weeks.

According to a particularly preferred variant, the present invention is a process for dyeing keratin fibers, in particular human keratin fibers, preferably the hair, comprising:
i) the application to said fibers of a composition (G) comprising at least one alkoxysilane of formula (XVIII), oligomers thereof and/or mixtures thereof as described previously, and then
ii) optionally leaving said composition (G) on the fibers for 1 minute to 30 minutes, preferably 1 to 20 minutes, such as 10 minutes, and then
iii) optional rinsing of said fibers, and then
iv) the application to said fibers of a composition (F) comprising:
a) at least one alkoxysilane of formula (I), oligomers thereof and/or mixtures thereof as described previously,
b) at least one alkoxysilane of formula (II), oligomers thereof and/or mixtures thereof as described previously,
c) at least one nonamino silicone of formula (III) as described previously,
d) at least one coloring agent chosen from pigments, direct dyes and mixtures thereof, and
e) optionally water, and then
v) an optional heating step as described previously, said heating possibly being continuous or fractionated.

According to a particularly preferred variant, the process for dyeing keratin fibers, in particular human keratin fibers, preferably the hair, comprises:
i) the application to said fibers of a composition (G) comprising at least one alkoxysilane of formula (XVIII), oligomers thereof and/or mixtures thereof as described previously, and then
ii) optionally leaving said composition (G) on the fibers for 1 minute to 30 minutes, preferably 1 to 20 minutes, such as 10 minutes, and then
iii) optional rinsing of said fibers, and then
iv) the application to said fibers of a composition (F) resulting from the extemporaneous mixing:
a) of a composition (A) containing at least one alkoxysilane of formula (I), oligomers thereof and/or mixtures thereof as described previously, at least one alkoxysilane of formula (II), oligomers thereof and/or mixtures thereof as described previously, and water, the pH of said composition (A) preferably being between 2.5 and 5.5;
b) of a composition (B) containing at least one nonamino silicone of formula (III) as described previously and at least one coloring agent chosen from pigments, direct dyes and mixtures thereof, and
v) a heating step as described previously, said heating possibly being continuous or fractionated.

Preferably, the alkoxysilane(s) of formula (I), oligomers thereof and/or a mixture thereof, the alkoxysilane(s) of formula (II), oligomers thereof and/or a mixture thereof and the nonamino silicone(s) of formula (III) included in the dye composition are identical or different in each step of repetition of the dyeing process.

The present invention also relates to a device for treating human keratin fibers such as the hair, comprising two compartments containing:
in a first compartment (E1), a composition (A) comprising at least one alkoxysilane of formula (I), oligomers thereof and/or mixtures thereof as described previously and at least one alkoxysilane of formula (II), oligomers thereof and/or mixtures thereof as described previously, and optionally water;
in a second compartment (E2), a composition (B) comprising at least one nonamino silicone of formula (III) as described previously;
it being understood that composition (A) and/or composition (B) optionally comprise at least one coloring agent chosen from pigments, direct dyes and mixtures thereof.

According to a preferred variant of the invention, the treating device according to the invention is a device for treating human keratin fibers such as the hair, comprising three compartments containing:
in a first compartment (E1), a composition (G) containing at least one alkoxysilane of formula (XVIII), oligomers thereof and/or mixtures thereof as described previously;
in a second compartment (E2), a composition (A) comprising at least one alkoxysilane of formula (I), oligomers thereof and/or mixtures thereof as described previously and at least one alkoxysilane of formula (II), oligomers thereof and/or mixtures thereof as described previously, and optionally water;
in a third compartment (E3), a composition (B) comprising at least one nonamino silicone of formula (III) as described previously;
it being understood that composition (A) and/or composition (B) optionally comprise at least one coloring agent chosen from pigments, direct dyes and mixtures thereof.

The present invention will now be described more specifically by means of examples, which do not in any way limit the scope of the invention. However, the examples make it possible to support specific features, variants and preferred embodiments of the invention.

### EXAMPLES

In the examples, the temperature is given in degrees Celsius and corresponds to room temperature (20-25° C.), unless otherwise indicated, and the pressure is atmospheric pressure, unless otherwise indicated.

### Example 1: Measurement of the Contact Angle

In order to check the presence of a deposit at the surface of the hair, a contact angle measurement was performed on N90W (natural, 90% white) hair treated with a composition according to the invention, and compared with untreated N90W (natural, 90% white) hair.

### Protocol for Measuring the Contact Angle

Hair strands mounted on supports are placed in contact with the surface of a liquid. The wetting force generated by the contact between the hair and the liquid is measured. This force varies according to the affinity of the hair for the liquid. Measurement of this force makes it possible to assess the surface state of the hair strands.

The hair strands are mounted on the support with the root upward and the end downward. Penetration is thus performed against the direction of lie of the scales.

The measuring machine used for the measurement is a Krüss K100SF tensiometer. The container containing the wetting liquid (water) is moved to come into contact with the solid sample (the hair).

The surface tension of the wetting liquid is measured with a platinum knife.

By using the equation for the knife method, the contact angle θ may be determined on establishing the value for the surface tension of ultrapure water, the wetting perimeter L and the measured force F, according to the following formula:

$$cos θ = \frac{F}{L ⋅ σ}$$

In general, a contact angle of less than 90 ° indicates that the wettability of the surface is very favorable: the liquid then spreads over a large area of solid. Conversely, a contact angle of greater than 90 ° generally indicates that the wettability of the surface is unfavorable: the liquid will then minimize the contact with the surface and form a compact drop of liquid.

For water, a wettable surface is said to be hydrophilic, whereas a non-wettable surface is said to be hydrophobic.

Measurement of the contact angle of the untreated hair was performed on nine different hair strands and a mean was calculated.

Measurement of the contact angle of the treated hair using the composition according to the invention was performed on ten different hair strands and a mean was calculated.

The treated hair strands were treated with the composition according to the invention according to the protocol described below.

### Hair Treatment Protocol

Composition P is prepared according to the following process: pure TEOS (reference 333859 sold by the company Sigma-Aldrich) and n-octyltriethoxysilane (reference 440213 sold by the company Sigma-Aldrich) are mixed with a solution of ethanol at pH = 3-4 and water, and said mixture is placed on a VWR brand magnetic stirrer (rotation speed 500 rpm) for 6 hours at a temperature of 50° C.

Composition Q is prepared according to the following process: the polydimethylsiloxane (PDMS) compound is diluted in an ethanol solution in which the pigment, present as tracer, is dispersed.

Compositions P and Q are vortex-mixed and the mixture of compositions P and Q, referred to as composition R, is applied to the locks of N90W (natural, 90% white) hair with a bath ratio of 0.75 g of composition per gram of hair.

The locks of hair are left for 5 minutes at room temperature before being dried with a hairdryer. After leaving to stand for one hour at room temperature, the locks of hair are placed in an oven at a temperature of 120° C. for 3 hours.

The various compounds are present in the amounts below (expressed in g/100 g).

TABLES 1

| | |
|---|---|
| Chemical name | Composition R |
| Tetraethoxysilane (TEOS, reference 333859 sold by the company Sigma-Aldrich ) | 14 |
| N-Octyltriethoxysilane (reference 440213 sold by the company Sigma-Aldrich ) | 33.5 |
| Polydimethylsiloxanes (PDMS) bearing hydroxyl end functions (reference DMS-S31 sold by the company Gelest) | 12 |
| Pigment (SunPURO™ Red Iron Oxide (C33-8001)) | 2 |
| Water | 5.7 |
| Ethanol | qs |

### Result

Table 2 corresponds to the mean and standard deviation values for the entry contact angle measurements as a function of the depth for untreated N90W hair.

TABLES 2

| Depth (in mm) | Contact angle Mean (in °) | Contact angle Standard deviation |
|---|---|---|
| 0 | 92.67 | 8.59 |
| 0.05 | 94.67 | 9.71 |
| 0.1 | 94.94 | 6.84 |
| 0.15 | 95.26 | 6.50 |
| 0.2 | 94.18 | 6.20 |
| 0.25 | 93.51 | 6.64 |
| 0.3 | 92.85 | 6.84 |
| 0.35 | 94.42 | 7.01 |
| 0.4 | 95.20 | 7.32 |
| 0.45 | 94.73 | 7.39 |
| 0.5 | 94.35 | 6.75 |
| 0.55 | 94.50 | 7.18 |
| 0.6 | 94.17 | 7.03 |
| 0.65 | 94.49 | 6.65 |
| 0.7 | 95.62 | 6.87 |
| 0.75 | 95.00 | 8.83 |
| 0.8 | 94.56 | 7.71 |
| 0.85 | 95.03 | 7.18 |
| 0.9 | 95.08 | 7.55 |
| 0.95 | 95.17 | 7.70 |
| 1 | 95.53 | 7.51 |
| 1.05 | 95.65 | 7.65 |
| 1.10 | 96.88 | 7.03 |
| 1.15 | 96.51 | 6.62 |
| 1.2 | 96.85 | 5.78 |
| 1.25 | 97.07 | 7.12 |
| 1.3 | 97.02 | 7.11 |
| 1.35 | 96.22 | 7.62 |
| 1.4 | 96.50 | 7.44 |
| 1.45 | 95.88 | 8.73 |
| 1.5 | 95.92 | 7.35 |
| 1.55 | 95.11 | 9.45 |
| 1.6 | 95.06 | 9.70 |
| 1.65 | 96.04 | 7.93 |
| 1.7 | 94.88 | 8.94 |
| 1.75 | 95.99 | 8.86 |
| 1.8 | 96.62 | 9.00 |
| 1.85 | 97.18 | 7.82 |
| 1.9 | 93.66 | 7.83 |

Table 3 corresponds to the mean and standard deviation values for the entry contact angle measurements as a function of the depth for N90W hair treated with composition R according to the invention.

TABLES 3

| Depth (in mm) | Contact angle Mean (in °) | Contact angle Standard deviation |
|---|---|---|
| 0.02 | 103.05 | 2.11 |
| 0.05 | 111.08 | 4.14 |
| 0.1 | 118.60 | 9.50 |
| 0.15 | 113.83 | 7.25 |
| 0.2 | 116.73 | 8.82 |
| 0.25 | 115.14 | 6.73 |
| 0.3 | 114.27 | 7.97 |
| 0.36 | 115.33 | 8.54 |
| 0.4 | 11.73 | 6.25 |
| 0.46 | 116.31 | 7.42 |
| 0.47 | 114.43 | 7.41 |
| 0.48 | 113.76 | 5.90 |
| 0.49 | 114.06 | 6.63 |
| 0.51 | 115.15 | 6.37 |
| 0.55 | 114.53 | 5.80 |
| 0.59 | 113.96 | 6.78 |
| 0.65 | 113.88 | 7.28 |
| 0.7 | 113.50 | 7.46 |
| 0.76 | 113.56 | 6.35 |
| 0.8 | 113.16 | 4.91 |
| 0.85 | 113.15 | 6.04 |
| 0.9 | 114.28 | 7.11 |
| 0.95 | 113.36 | 9.02 |
| 1 | 113.21 | 6.92 |
| 1.05 | 112.26 | 9.84 |
| 1.1 | 114.05 | 7.71 |
| 1.16 | 112.77 | 7.43 |
| 1.2 | 113.77 | 6.92 |
| 1.25 | 111.83 | 5.18 |
| 1.3 | 113.95 | 3.86 |
| 1.36 | 114.84 | 5.22 |
| 1.4 | 112.74 | 6.00 |
| 1.45 | 112.58 | 6.26 |
| 1.5 | 115.81 | 6.51 |
| 1.55 | 115.32 | 7.47 |
| 1.6 | 116.07 | 8.15 |
| 1.65 | 114.77 | 7.28 |
| 1.71 | 113.11 | 5.12 |
| 1.75 | 114.04 | 6.42 |
| 1.8 | 112.77 | 6.12 |
| 1.85 | 114.53 | 5.95 |
| 1.88 | 113.47 | 5.81 |

The contact angle values greater than 90° for the locks of hair treated with composition R according to the invention confirm the presence of a hydrophobic deposit on the surface of the treated hair.

In order to evaluate the persistence of the deposit with respect to shampoo washing, the hair was subjected to a cycle of three successive shampoo washes, the locks of hair are first combed and each shampoo wash then comprises the following steps: the locks are washed using a standard shampoo (Garnier Ultra Doux), and then rinsed.

At the end of the last shampoo wash, the locks of hair are combed and dried with a hairdryer.

The contact angles were again measured and the following results were obtained.

Table 4 corresponds to the mean and standard deviation values for the entry contact angle measurements as a function of the depth for untreated N90W hair and after three shampoo washes.

TABLES 4

| Depth (in mm) | Contact angle Mean (in °) | Contact angle Standard deviation |
|---|---|---|
| 0 | 83.67 | 5.64 |
| 0.05 | 88.74 | 5.42 |
| 0.1 | 89.59 | 6.88 |
| 0.15 | 88.70 | 6.89 |
| 0.2 | 88.65 | 6.52 |
| 0.25 | 88.57 | 7.08 |
| 0.3 | 88.43 | 7.11 |
| 0.35 | 88.28 | 6.05 |
| 0.4 | 88.97 | 6.16 |
| 0.45 | 88.34 | 6.12 |
| 0.5 | 89.43 | 5.29 |
| 0.55 | 90.24 | 5.96 |
| 0.6 | 89.50 | 5.33 |
| 0.65 | 89.34 | 6.13 |
| 0.7 | 89.51 | 5.69 |
| 0.75 | 89.70 | 6.74 |
| 0.8 | 88.58 | 6.66 |
| 0.85 | 88.42 | 6.00 |
| 0.91 | 88.24 | 6.08 |
| 0.95 | 88.80 | 6.01 |
| 1 | 88.96 | 6.33 |
| 1.05 | 89.10 | 5.15 |
| 1.06 | 88.77 | 5.38 |
| 1.10 | 87.98 | 5.50 |
| 1.15 | 88.68 | 7.61 |
| 1.2 | 88.25 | 6.75 |
| 125 | 88.75 | 7.12 |
| 1.3 | 88.20 | 6.28 |
| 1.36 | 88.22 | 6.12 |
| 1.40 | 88.61 | 5.27 |
| 1.45 | 88.80 | 4.12 |
| 1.50 | 88.45 | 5.35 |
| 1.55 | 88.82 | 5.82 |
| 1.60 | 90.56 | 6.90 |
| 1.65 | 86.67 | 10.91 |
| 1.71 | 87.17 | 10.53 |
| 1.75 | 87.90 | 9.49 |
| 1.80 | 88.58 | 9.10 |
| 1.85 | 87.58 | 8.55 |
| 1.9 | 87.10 | 8.56 |

Table 5 corresponds to the mean and standard deviation values for the entry contact angle measurements as a function of the depth for N90W hair treated with composition R according to the invention after three shampoo washes.

TABLES 5

| Depth (in mm) | Contact angle Mean (in °) | Contact angle Standard deviation |
|---|---|---|
| 0.00 | 98.46 | 7.88 |
| 0.05 | 108.53 | 10.46 |
| 0.1 | 112.03 | 10.15 |
| 0.15 | 112.07 | 10.33 |
| 0.2 | 114.57 | 12.47 |
| 0.25 | 111.36 | 8.22 |
| 0.3 | 113.45 | 9.79 |
| 0.35 | 113.42 | 10.68 |
| 0.4 | 110.60 | 8.75 |
| 0.45 | 112.01 | 9.28 |
| 0.50 | 108.47 | 8.57 |
| 0.55 | 111.30 | 10.36 |
| 0.60 | 110.15 | 10.60 |
| 0.65 | 110.81 | 8.73 |
| 0.70 | 109.27 | 8.27 |
| 0.75 | 111.17 | 11.41 |
| 0.80 | 110.85 | 9.81 |
| 0.85 | 111.87 | 9.91 |
| 0.91 | 109.26 | 9.59 |
| 0.95 | 114.23 | 12.08 |
| 1.00 | 110.74 | 9.61 |
| 1.05 | 110.73 | 9.21 |
| 1.10 | 112.23 | 11.34 |
| 1.15 | 109.46 | 10.14 |
| 1.20 | 112.05 | 10.25 |
| 1.25 | 113.92 | 15.06 |
| 1.30 | 110.59 | 10.21 |
| 1.36 | 109.49 | 9.10 |
| 1.40 | 108.12 | 9.00 |
| 1.45 | 110.32 | 12.79 |
| 1.50 | 111.42 | 13.33 |
| 1.55 | 111.71 | 14.28 |
| 1.60 | 111.04 | 10.13 |
| 1.65 | 110.98 | 10.57 |
| 1.71 | 111.04 | 9.93 |
| 1.75 | 112.85 | 11.36 |
| 1.80 | 110.60 | 9.30 |
| 1.85 | 110.32 | 8.71 |
| 1.90 | 111.00 | 10.76 |

The contact angle values for the untreated hair after three shampoo washes are lower than those obtained for the hair treated with composition R and which has undergone three shampoo washes.

The contact angle values greater than 90 ° for the locks of hair treated with composition R and which have undergone three shampoo washes confirm the presence of a hydrophobic deposit, which is persistent with respect to shampoo washing, on the surface of the treated hair.

Thus, treatment of the hair with composition R according to the invention leads to hydrophobization of the keratin fibers which makes them less sensitive to humidity and thus to the appearance of frizziness in a humid environment, and this hydrophobization is persistent with respect to shampoo washing.

### Example 2

Solution 1 (i.e. solution of tetraethoxysilane (TEOS) and of n-octyltriethoxysilane) is prepared according to the process below:
pure TEOS (reference 333859 sold by the company Sigma-Aldrich) and n-octyltriethoxysilane (reference 440213 sold by the company Sigma-Aldrich) are mixed with a solution of ethanol at pH = 3-4 and water, and said mixture is placed on a VWR brand magnetic stirrer (rotation speed 500 rpm) for 6 hours at a temperature of 50° C.

Solution 2 (i.e. solution of nonamino silicone) is prepared according to the process below:
the polydimethylsiloxane (PDMS) compound bearing trialkoxysilane end functions (PDMS reference Silmer TMS Di-10 sold by the company Siltech) or the polydimethylsiloxane (PDMS) compound bearing hydroxyl end functions (PDMS reference DMS-S31 sold by the company Gelest) is diluted in an ethanol solution in which the pigment (iron oxide sold by the company Sun Chemical under the name SunPuro™ Red Iron Oxide (C33-8001)) is dispersed.

Solution 1 is mixed with solution 2 and said mixture (composition A or composition B depending on the nature of the PDMS used) is applied to the keratin fibers.

The various compounds are present in the amounts below (expressed in g/100 g).

TABLES 6

| | | |
|---|---|---|
| Chemical name | Composition A | Composition B |
| Tetraethoxysilane (TEOS) | 13.9 | 13.9 |
| N-Octyltriethoxysilane | 3.5 | 3.5 |
| Polydimethylsiloxanes (PDMS) bearing trialkoxysilane end functions | 12 | - |
| Polydimethylsiloxanes (PDMS) bearing hydroxyl end functions | - | 12 |
| Pigment (iron oxide sold by the company Sun Chemical under the name SunPuro™ Red Iron Oxide (C33-8001)) | 3 | 3 |
| Water | 5.7 | 5.7 |
| Ethanol | qs | qs |

### Protocol

Composition A and composition B according to the invention are applied to locks of dry natural hair containing 90% white hairs, at a rate of 1 g of composition per gram of lock.

The locks of hair are then combed and dried with a hairdryer.

The locks of hair are left for two weeks at room temperature and are then placed in an oven at a temperature of 120° C. for 10 hours.

The hair is dyed uniformly and intensely.

The locks of hair thus dyed are then subjected to a test of several repeated shampoo washes so as to evaluate the fastness (persistence) of the coloring obtained with respect to shampoo washing.

### Shampoo Wash Protocol

The locks are washed with a standard shampoo (Garnier Ultra Doux).

The locks of hair are then rinsed, combed and dried with a hairdryer.

The next shampoo wash is performed on the locks obtained after the application of the hairdryer.

### Results

The persistence of the color of the locks was evaluated in the CIE L* a* b* system, using a Minolta Spectrophotometer CM3600D colorimeter (illuminant D65, angle 10 °, specular component included).

In this L*a*b* system, L* represents the intensity of the color, a* indicates the green/red color axis and b* the blue/yellow color axis.

The persistence of the coloring is evaluated by the color difference ΔE between the dyed locks before shampooing, then after having undergone one and three shampoo washes according to the protocol described above. The lower the ΔE value, the more persistent the color with respect to shampoo washing.

The ΔE value is calculated according to the following equation:

$$Δ \text{E} = \sqrt{{\left(\text{L*} - {\text{L}}_{\text{o}} *\right)}^{2} + {\left(\text{a*} - {\text{a}}_{\text{o}} *\right)}^{2} + {\left(\text{b*} - {\text{b}}_{\text{o}} *\right)}^{2}}$$

In this equation, L*a*b* represent the values measured after dyeing the hair and after performing the shampoo washes, and L₀*a₀*b₀* represent the values measured after dyeing the hair but before shampoo washing.

TABLES 7

| Compositions | Number of shampoo washes | L* | a* | b* | ΔE |
|---|---|---|---|---|---|
| Composition A | 0 | 30.70 | 34.83 | 24.85 | 0 |
| | 3 | 33.74 | 31.69 | 21.05 | 5.79 |
| Composition B | 0 | 35.47 | 33.91 | 24.75 | 0 |
| | 3 | 38.30 | 34.12 | 25.52 | 2.94 |

The locks of hair dyed with composition A according to the invention or composition B according to the invention and washed with three shampoo washes have low ΔE values.

Thus, the coloring of the keratin fibers obtained using the compositions according to the invention shows good persistence with respect to shampoo washing. Specifically, the locks of hair dyed with the compositions according to the invention and washed with three shampoo washes show good persistence of the color.

### Example 3: Pretreatment With APTES

Solution 1 (i.e. solution of tetraethoxysilane (TEOS) and of n-octyltriethoxysilane) is prepared according to the process below:
pure TEOS (reference 333859 sold by the company Sigma-Aldrich) and n-octyltriethoxysilane (reference 440213 sold by the company Sigma-Aldrich) are mixed with a solution of ethanol at pH = 3-4 and water, and said mixture is placed on a VWR brand magnetic stirrer (rotation speed 500 rpm) for 6 hours at a temperature of 50° C.

Solution 2 (i.e. solution of nonamino silicone) is prepared according to the process below:
the polydimethylsiloxane (PDMS) compound bearing hydroxyl end functions (PDMS reference DMS-S31 sold by the company Gelest) or the polydimethylsiloxane (PDMS) compound bearing trialkoxysilane end functions (PDMS reference Silmer TMS Di-10 sold by the company Siltech) is diluted in an ethanol solution in which the pigment (iron oxide sold by the company Sun Chemical under the name SunPuro™ Red Iron Oxide (C33-8001)) is dispersed.

Solution 1 is mixed with solution 2 and said mixture (composition C or composition D depending on the nature of the nonamino silicone used) is applied to the keratin fibers.

The various compounds are present in the amounts below (expressed in g/100 g).

TABLES 8

| | | |
|---|---|---|
| Chemical name | Composition C | Composition D |
| Tetraethoxysilane (TEOS) | 13.9 | 13.9 |
| N-Octyltriethoxysilane | 3.5 | 3.5 |
| Polydimethylsiloxanes (PDMS) bearing hydroxyl end functions | 12 | - |
| Polydimethylsiloxanes (PDMS) bearing trialkoxysilane end functions | - | 12 |
| Pigment (iron oxide sold by the company Sun Chemical under the name SunPuro™ Red Iron Oxide (C33-8001) | 3 | 3 |
| Water | 5.9 | 5.7 |
| Ethanol | qs | qs |

### Protocol

Before applying composition C or composition D to the keratin fibers, the locks of natural hair containing 90% white hairs are pretreated with a solution 3 which is prepared according to the following process:

3.66 g of 0.1 N HCl are added to 30 g of APTES (APTES Silsoft A- 1100 sold by the company Momentive Performance Materials) in 200 g of water. The solution obtained is stirred at room temperature for 2 hours. Once the hydrolysis is complete, the solution is transferred into a beaker containing 20 locks of natural hair containing 90% white hairs, and left under gentle stirring for 10 minutes. Once the impregnation is complete, the locks of hair are rinsed and left to dry between two sheets of absorbent paper.

Compositions C and D according to the invention are applied to dry pretreated locks of natural hair containing 90% white hairs, at a rate of 1 g of composition per gram of lock.

The locks of hair are then combed and dried with a hairdryer.

The locks of hair are left for two weeks at room temperature and are then placed in an oven at a temperature of 120° C. for 10 hours.

The hair is dyed uniformly and intensely.

The locks of hair thus dyed are then subjected to a test of several repeated shampoo washes so as to evaluate the fastness (persistence) of the coloring obtained with respect to shampoo washing.

The shampoo wash protocol is identical to that presented in Example 2.

### Results

The persistence of the color of the locks was evaluated in the CIE L* a* b* system, using a Minolta Spectrophotometer CM3600D colorimeter (illuminant D65, angle 10 °, specular component included), as explained in Example 1.

TABLES 9

| Compositions | Number of shampoo washes | L* | a* | b* | ΔE |
|---|---|---|---|---|---|
| Composition C | 0 | 33.08 | 36.91 | 26.45 | 0 |
| | 3 | 32.32 | 34.72 | 24.58 | 2.98 |
| Composition D | 0 | 28.16 | 34.61 | 25.12 | 0 |
| | 3 | 28.93 | 34.54 | 23.19 | 2.08 |

The locks of hair pretreated with a solution of aminoalkoxysilane and dyed with compositions C or D according to the invention and washed with three shampoo washes have low ΔE values.

Thus, the coloring of the keratin fibers obtained using the compositions according to the invention shows good persistence with respect to shampoo washing. Specifically, the locks of hair dyed with the compositions according to the invention and washed with three shampoo washes show good persistence of the color.

### Example 4

Solution 1 (i.e. solution of tetraethoxysilane (TEOS) and of n-octyltriethoxysilane) is prepared according to the process below:
pure TEOS (reference 333859 sold by the company Sigma-Aldrich) and n-octyltriethoxysilane (reference 440213 sold by the company Sigma-Aldrich) are mixed with a solution of ethanol at pH = 3-4 and water, and said mixture is placed on a VWR brand magnetic stirrer (rotation speed 500 rpm) for 6 hours at a temperature of 50° C.

Solution 2 (i.e. solution of nonamino silicone) is prepared according to the process below:
the polydimethylsiloxane (PDMS) compound bearing trialkoxysilane end functions (PDMS reference Silmer TMS Di-10 sold by the company Siltech) or the polydimethylsiloxane (PDMS) compound bearing hydroxyl end functions (PDMS reference DMS-S31 sold by the company Gelest) is diluted in an ethanol solution in which the pigment (iron oxide sold by the company Sun Chemical under the name SunPuro™ Red Iron Oxide (C33-8001)) is dispersed.

Solution 1 is mixed with solution 2 and said mixture (composition E or F depending on the nature of the nonamino silicone used) is applied to the keratin fibers.

Solution 1 alone is also applied, according to the same protocol, to the keratin fibers (composition G).

The various compounds are present in the amounts below (expressed in g/100 g).

TABLES 10

| | | | |
|---|---|---|---|
| Chemical name | Composition E (Invention) | Composition F (Invention) | Composition G (comparative) |
| Tetraethoxysilane (TEOS) | 14 | 14 | 14 |
| N-Octyltriethoxysilane | 3.5 | 3.5 | 3.5 |
| Polydimethylsiloxanes (PDMS) bearing hydroxyl end functions | 12 | - | - |
| Polydimethylsiloxanes (PDMS) bearing trialkoxysilane end functions | - | 12 | - |
| Pigment | 2 | 2 | 2 |
| Water | 5.7 | 5.7 | 5.7 |
| Ethanol | qs | qs | qs |

### Protocol

Compositions E and F according to the invention and also comparative composition G are applied to locks of dry natural hair containing 90% white hairs, at a rate of 1 g of composition per gram of lock.

The locks of hair are then combed and dried with a hairdryer.

The locks of hair are left for 3 days at room temperature.

The locks of hair thus dyed are then subjected to a test of several repeated shampoo washes so as to evaluate the fastness (persistence) of the coloring obtained with respect to shampoo washing.

The shampoo wash protocol is identical to that presented in Example 2.

### Results

The persistence of the color of the locks was evaluated in the CIE L* a* b* system, using a Minolta Spectrophotometer CM3600D colorimeter (illuminant D65, angle 10 °, specular component included), as explained in Example 1.

TABLES 11

| Compositions | Number of shampoo washes | L* | a* | b* | ΔE |
|---|---|---|---|---|---|
| Composition E (Invention) | 0 | 37.18 | 28.78 | 18.39 | 0 |
| | 1 | 39.20 | 28.97 | 19.88 | 2.52 |
| | 3 | 49.03 | 24.74 | 22.31 | 13.12 |
| Composition F (invention) | 0 | 34.20 | 31.02 | 18.72 | 0 |
| | 1 | 37.46 | 30.50 | 20.17 | 3.61 |
| | 3 | 45.50 | 27.41 | 23.12 | 12.65 |
| Composition G (comparative) | 0 | 35.29 | 33.51 | 20.39 | 0 |
| | 1 | 39.64 | 30.53 | 19.82 | 5.30 |
| | 3 | 52.30 | 21.83 | 17.11 | 20.89 |

The locks of hair dyed with compositions E and F according to the invention and washed with one or three shampoo washes have lower ΔE values than the locks of hair dyed with comparative composition G.

Thus, the coloring of the keratin fibers obtained using the compositions according to the invention shows good persistence with respect to shampoo washing. Specifically, the locks of hair dyed with the compositions according to the invention and washed with one or three shampoo washes show good persistence of the color.

### Example 5: Pretreatment With APTES

### Protocol

Before applying composition E to the keratin fibers (composition described in example 4), the locks of hair are pretreated with a solution 3 as described in example 3.

Composition E according to the invention is applied to pretreated locks of dry natural hair containing 90% white hairs, at a rate of 1 g of composition per gram of lock.

The locks of hair are then combed and dried with a hairdryer.

The locks of hair are left for three days at room temperature.

The hair is dyed uniformly and intensely.

The locks of hair thus dyed are then subjected to a test of several repeated shampoo washes so as to evaluate the fastness (persistence) of the coloring obtained with respect to shampoo washing.

The shampoo wash protocol is identical to that presented in Example 2.

### Results

The persistence of the color of the locks was evaluated in the CIE L* a* b* system, using a Minolta Spectrophotometer CM3600D colorimeter (illuminant D65, angle 10 °, specular component included), as explained in Example 1.

TABLES 12

| Compositions | Number of shampoo washes | L* | a* | b* | ΔE |
|---|---|---|---|---|---|
| Composition E + pretreatment with APTES | 0 | 36.03 | 29.34 | 19.28 | 0 |
| | 1 | 39.13 | 29.60 | 20.56 | 3.36 |
| | 3 | 40.41 | 30.01 | 22.89 | 5.72 |

The locks of hair pretreated with a solution of aminoalkoxysilane, dyed with composition E according to the invention and washed with one or three shampoo washes have low ΔE values.

Thus, the coloring of the keratin fibers which is obtained using the composition according to the invention shows good persistence with respect to shampoo washing. Specifically, the locks of hair dyed wih the compositions according to the invention and washed with one or three shampoo washes show good persistence of the color.

### Example 6 : Hair Coloration With and Without TEOS

The following compositions were prepared according to the protocols disclosed in the examples below The various compounds are present in the amounts below (expressed in g/100 g and active ingredients).

TABLES 13

| | | |
|---|---|---|
| Chemical name | Composition 6 (Invention) | Composition 6C (Comparison) |
| Tetraethoxysilane (TEOS) | 14 | - |
| n-Octyltriethoxysilane | 3.5 | |
| Polydimethylsiloxanes (PDMS) bearing hydroxyl end functions | 12 | |
| Pigment | 2 | |
| Water | 5.7 | |
| Ethanol | Qsp 100 g | |

Compositions 6 according to the invention and also comparative composition 6C are applied to locks of dry natural hair containing 90% white hairs, and also to locks of bleached hair, at a rate of 1 g of composition per gram of lock.

The locks of hair are then combed and dried with a hairdryer.

The locks of hair are left for 3 days at room temperature.

The locks of hair thus dyed are then subjected to a test of several repeated shampoo washes so as to evaluate the fastness (persistence) of the coloring obtained with respect to shampoo washing.

### Shampoo Wash Protocol

The locks are washed with a standard shampoo (DOP).

The locks of hair are then rinsed, combed and dried with a hairdryer.

The next shampoo wash is performed on the locks obtained after the application of the hairdryer.

### Results

The persistence of the color of the locks was evaluated in the CIE L* a* b* system, using a Minolta Spectrophotometer CM3600D colorimeter (illuminant D65, angle 10 °, specular component included), as explained in Example 1.

The color fastness evaluation was as followed:

TABLES 14

Natural Hair with 90% white hair

| Compositions | Number of shampoos | L* | a* | b* | ΔE |
|---|---|---|---|---|---|
| Composition 6 (Invention) | 0 | 39.1 | 27.01 | 22.06 | 0 |
| | 1 | 39.82 | 22.28 | 19.62 | 5.4 |
| | 3 | 40.98 | 20.39 | 18.51 | 7.744 |
| Composition 6C (comparison) | 0 | 40.27 | 24.35 | 20.98 | 0 |
| | 1 | 51.97 | 10.76 | 16.7 | 18.444 |
| | 3 | 57.41 | 7.91 | 20.08 | 23.8 |

TABLES 14

Bleached hair

| Compositions | Number of shampoos | L* | a* | b* | ΔE |
|---|---|---|---|---|---|
| Composition 6 (Invention) | 0 | 38.17 | 24.28 | 27.83 | 0 |
| | 1 | 45.84 | 22.15 | 27.08 | 8.0 |
| | 3 | 48.88 | 18.98 | 26.39 | 12.0 |
| Composition 6C (comparison) | 0 | 35.36 | 29.43 | 26.69 | 0 |
| | 1 | 57.79 | 9.82 | 28.05 | 29.8 |
| | 3 | 59.37 | 8.18 | 26.51 | 32.1 |

These results show that the color retention is improved with the composition of the invention over the same composition but without TEOS. The same improvement is obtained on nature and bleached hair.

1-22. (canceled)

23. A composition (F) for treating keratin fibers comprising:
a) at least one alkoxysilane chosen from compounds of formula (I) below, oligomers thereof, or mixtures thereof: 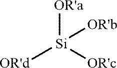
in which:
R′a, R′b, R′c, and R’d are independently chosen from alkyl groups containing from 1 to 10 carbon atoms;
b) at least one alkoxysilane chosen from compounds of formula (II) below, oligomers thereof, or mixtures thereof: 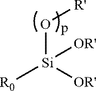
in which:
R₀ is chosen from alkyl groups containing from 4 to 24 carbon atoms, optionally interrupted with a dialkylammonium divalent radical RₐR_{b}N+An⁻, with:
Rₐ and R_{b} independently chosen from alkyl groups containing from 1 to 10 carbon atoms, and
An⁻ represents a cosmetically acceptable anion chosen from fluoride, chloride, bromide, or iodide;
R′ is independently chosen from a hydrogen atom or alkyl groups containing from 1 to 20 carbon atoms; and
p denotes an integer ranging from 0 to 1;
c) at least one non-amino silicone chosen from compounds of formula (III) below: 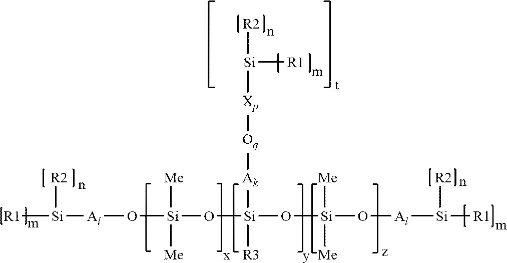
in which:
R1 is independently chosen from hydroxyl groups, alkoxy groups containing from 1 to 2 carbon atoms, or alkyl groups containing from 1 to 2 carbon atoms;
R2 is independently chosen from alkyl groups containing from 1 to 20 carbon atoms;
R3 is chosen from a hydroxyl group, an alkyl group containing from 1 to 10 carbon atoms, said alkyl group optionally substituted with at least one group chosen from a hydroxyl group (OH) or a thiol group (SH), a cycloalkyl group containing from 3 to 20 carbon atoms, said cycloalkyl optionally substituted with at least one group chosen from a hydroxyl group (OH) or a thiol group (SH), an alkoxy group containing from 1 to 2 carbon atoms, said alkoxy group optionally substituted with at least one group chosen from a hydroxyl group (OH) or a thiol group (SH), an aryl group containing from 6 to 12 carbon atoms, said aryl group optionally substituted with at least one group chosen from a hydroxyl group (OH) or a thiol group (SH), or a radical —(O)p′—Si(R₁)m′(R₂)n′;
A is independently chosen from saturated divalent hydrocarbon-based radicals containing from 1 to 2 carbon atoms,
X is chosen from a hydrogen atom or a saturated divalent hydrocarbon-based radical containing from 1 to 2 carbon atoms;
m independently denotes an integer ranging from 1 to 3;
n independently denotes an integer ranging from 0 to 2;
m′ independently denotes an integer ranging from 1 to 3;
n′ independently denotes an integer ranging from 0 to 2;
p is an integer equal to 0, 1, 2, or 3;
p′ is an integer equal to 0 or 1;
k is an integer equal to 0, 1, 2, or 3;
q is an integer equal to 0 or 1;
j independently denotes an integer equal to 0 or 1;
t is an integer equal to 0 or 1;
y is an integer ranging from 0 to 10;
z is an integer ranging from 0 to 500;
m+n = 3;
m′+n′ = 3;
x+z ranges from 0 to 500; and
x+y+z ≥ 4;
with the proviso that:
if X represents a hydrogen atom, then t = 0 and p = 1, and
if p = 0, then t = 1 and q = 1, and that at least one of the radicals R₁ or
R₃ denotes a hydroxyl radical or an alkoxy radical containing from 1 to 2 carbon atoms; and
d) optionally at least one coloring agent chosen from pigments, direct dyes, or mixtures thereof.

24. The composition according to claim 23, wherein in formula (I), R′a, R′b, R′c, and R’d are identical and denote an ethyl radical.

25. The composition according to claim 23, comprising at least one alkoxysilane of formula (I) chosen from tetraethoxysilane (TEOS), tetrapropoxysilane, tetrabutoxysilane, tetrapentoxysilane, tetrahexyloxysilane, or mixtures thereof.

26. The composition according to claim 23, comprising a total amount of alkoxysilanes of formula (I) and oligomers thereof ranging from 0.1% to 30% by weight, relative to the total weight of composition (F).

27. The composition according to claim 23, wherein in formula (II):
R′ are identical and are chosen from alkyl groups containing from 1 to 4 carbon atoms, and
p is equal to 1.

28. The composition according to claim 27, wherein in formula (II), R₀ is chosen from an alkyl group containing from 6 to 18 carbon atoms.

29. The composition according to claim 23, comprising a total amount of alkoxysilanes of formula (II) and oligomers thereof ranging from 0.1% to 30% by weight, relative to the total weight of composition (F).

30. The composition according to claim 23, wherein in formula (III):
R2 is independently chosen from alkyl groups containing from 1 to 4 carbon atoms;
R3 is chosen from a hydroxyl group or an alkyl group containing from 1 to 10 carbon atoms;
j is equal to 0; and
k is equal to 0.

31. The composition according to claim 30, wherein in formula (III):
R2 is independently chosen from alkyl groups containing from 1 to 2 carbon atoms;
m denotes an integer ranging from 1 to 3;
n denotes an integer ranging from 0 to 2;
j is equal to 0;
- y is equal to 0;
m+n = 3; and
x+z ≥ 4;
wherein at least one of the radicals R1 denotes a hydroxyl radical or an alkoxy radical containing from 1 to 2 carbon atoms.

32. The composition according to claim 30, wherein the at least one non-amino silicone is chosen from compounds of formula (IIIa) below: 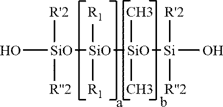
in which:
R₁ is independently chosen from a hydroxyl group, alkyl groups containing from 1 to 10 carbon atoms, alkoxy groups containing from 1 to 2 carbon atoms, or a group —(CH₂)ₛ—Si(R₄)₃ in which s denotes an integer ranging from 1 to 4 and R₄ is independently chosen from alkoxy radicals containing from 1 to 2 carbon atoms;
R′₂ and R″₂ are independently chosen from alkyl groups containing from 1 to 10 carbon atoms;
a denotes an integer ranging from 0 to 10;
b denotes an integer ranging from 0 to 500; and
a+b ≥ 4.

33. The composition according to claim 32, wherein in formula (IIIa):
R₁ is independently chosen from alkyl groups containing from 1 to 4 carbon atoms;
R′₂ and R″₂ are independently chosen from a hydroxyl group or alkyl groups containing from 1 to 4 carbon atoms;
a denotes an integer ranging from 0 to 5; and
b denotes an integer ranging from 0 to 10.

34. The composition according to claim 23, wherein the at least one non-amino silicone is chosen from compounds of formula (lllc) below: 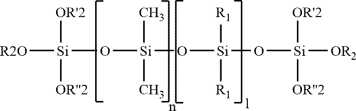
in which:
R₁ is independently chosen from alkyl groups containing from 1 to 10 carbon atoms, alkoxy groups containing from 1 to 2 carbon atoms, or a group —O—(X)ₚ—Si(OR₂)₃with X representing a saturated divalent hydrocarbon-based radical containing from 1 to 2 carbon atoms, and p denoting an integer ranging from 0 to 3;
R₂ is independently chosen from alkyl groups containing from 1 to 10 carbon atoms;
R′₂ and R″₂ are independently chosen from alkyl groups containing from 1 to 10 carbon atoms or alkoxy groups containing from 1 to 10 carbon atoms;
i denotes an integer ranging from 0 to 10;
n denotes an integer ranging from 0 to 500; and
n+i ranges from 0 to 510.

35. The composition according to claim 34, wherein in formula (lllc):
R₁ and R₂ are independently chosen from alkyl groups containing from 1 to 4 carbon atoms;
R′₂ and R″₂ are independently chosen from alkoxy groups containing from 1 to 4 carbon atoms; and
- n+i≥ 4.

36. The composition according to claim 23, comprising a total amount of non-amino silicones of formula (III) ranging from 0.1% to 25% by weight, relative to the total weight of composition (F).

37. The composition according to claim 23, wherein composition (F) comprises one or more organic solvent(s) chosen from C₁-C₄ lower alkanols.

38. A method for treating keratin fibers, comprising applying an effective amount of a composition (F) according to claim 23 to the keratin fibers.

39. A kit for treating keratin fibers comprising at least two compartments, wherein:
a first compartment (E1) comprises a composition (A) comprising:
a) at least one alkoxysilane chosen from compounds of formula (I) below, oligomers thereof, or mixtures thereof: 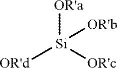
in which:
R′a, R′b, R′c, and R’d are independently chosen from alkyl groups containing from 1 to 10 carbon atoms; and
b) at least one alkoxysilane chosen from compounds of formula (II) below, oligomers thereof, or mixtures thereof: 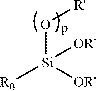
in which:
R₀ is chosen from alkyl groups containing from 4 to 24 carbon atoms, optionally interrupted with a dialkylammonium divalent radical RₐR_{b}N+An⁻, with:
Rₐ and R_{b} independently chosen from alkyl groups containing from 1 to 10 carbon atoms, and
An⁻ represents a cosmetically acceptable anion chosen from fluoride, chloride, bromide, or iodide;
R′ is independently chosen form a hydrogen atom or alkyl groups containing from 1 to 20 carbon atoms; and
p denotes an integer ranging from 0 to 1; and
- a second compartment (E2) comprises a composition (B) comprising:
c) at least one non-amino silicone chosen from compounds of formula (III) below: 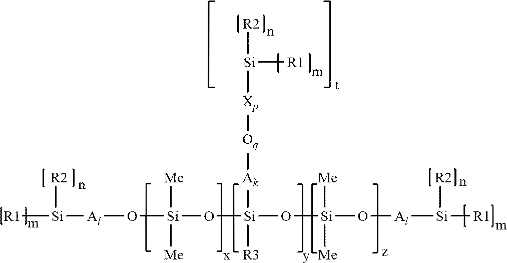
in which:
R1 is independently chosen from hydroxyl groups, alkoxy groups containing from 1 to 2 carbon atoms, or alkyl groups containing from 1 to 2 carbon atoms;
R2 is independently chosen from alkyl groups containing from 1 to 20 carbon atoms;
R3 is chosen from a hydroxyl group, an alkyl group containing from 1 to 10 carbon atoms, said alkyl group optionally substituted with at least one group chosen from a hydroxyl group (OH) or a thiol group (SH), a cycloalkyl group containing from 3 to 20 carbon atoms, said cycloalkyl optionally substituted with at least one group chosen from a hydroxyl group (OH) or a thiol group (SH), an alkoxy group containing from 1 to 2 carbon atoms, said alkoxy group optionally substituted with at least one group chosen from a hydroxyl group (OH) or a thiol group (SH), an aryl group containing from 6 to 12 carbon atoms, said aryl group optionally substituted with at least one group chosen from a hydroxyl group (OH) or a thiol group (SH), or a radical —(O)_{p’}—Si(R₁)_{m’}(R₂)_{n’};
A is independently chosen from saturated divalent hydrocarbon-based radicals containing from 1 to 2 carbon atoms,
X is chosen from a hydrogen atom or a saturated divalent hydrocarbon-based radical containing from 1 to 2 carbon atoms;
m independently denotes an integer ranging from 1 to 3;
n independently denotes an integer ranging from 0 to 2;
m′ independently denotes an integer ranging from 1 to 3;
n′ independently denotes an integer ranging from 0 to 2;
p is an integer equal to 0, 1, 2, or 3;
p′ is an integer equal to 0 or 1;
k is an integer equal to 0, 1, 2, or 3;
q is an integer equal to 0 or 1;
j independently denotes an integer equal to 0 or 1;
t is an integer equal to 0 or 1;
y is an integer ranging from 0 to 10;
z is an integer ranging from 0 to 500;
m+n = 3;
m′+n′ = 3;
x+z ranges from 0 to 500; and
x+y+z ≥ 4;
with the proviso that:
if X represents a hydrogen atom, then t = 0 and p = 1, and
if p = 0, then t = 1 and q = 1, and that at least one of the radicals R₁ or R₃ denotes a hydroxyl radical or an alkoxy radical containing from 1 to 2 carbon atoms;
wherein composition (A) and/or composition (B) optionally further comprise at least one coloring agent chosen from pigments, direct dyes, or mixtures thereof.

40. A process for treating keratin fibers which comprises mixing, at or about the time of use, compositions (A) and (B) to form a composition (F), and applying the composition (F) to the keratin fibers, with:
composition (A) comprising:
a) at least one alkoxysilane chosen from compounds of formula (l) below, oligomers thereof, or mixtures thereof: 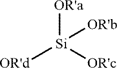
in which:
R′a, R′b, R′c, and R’d are independently chosen from alkyl groups containing from 1 to 10 carbon atoms; and
b) at least one alkoxysilane chosen from compounds of formula (II) below, oligomers thereof, or mixtures thereof: 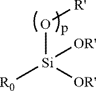
in which:
R₀ is chosen from alkyl groups containing from 4 to 24 carbon atoms, optionally interrupted with a dialkylammonium divalent radical RₐR_{b}N+An⁻, with:
Rₐ and R_{b} independently chosen from alkyl groups containing from 1 to 10 carbon atoms, and
An⁻ represents a cosmetically acceptable anion chosen from fluoride, chloride, bromide, or iodide;
R′ is independently chosen from a hydrogen atom or alkyl groups containing from 1 to 20 carbon atoms; and
p denotes an integer ranging from 0 to 1; and
composition (B) comprising:
c) at least one non-amino silicone chosen from compounds of formula (III) below: 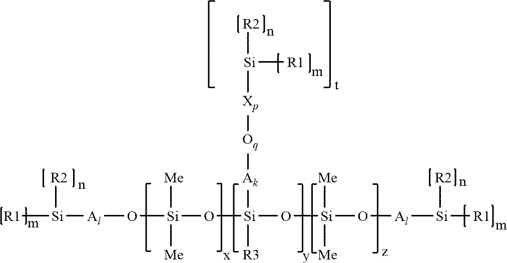
in which:
R1 is independently chosen from hydroxyl groups, alkoxy groups containing from 1 to 2 carbon atoms, or alkyl groups containing from 1 to 2 carbon atoms;
R2 is independently chosen from alkyl groups containing from 1 to 20 carbon atoms;
R3 is chosen from a hydroxyl group, an alkyl group containing from 1 to 10 carbon atoms, said alkyl group optionally substituted with at least one group chosen from a hydroxyl group (OH) or a thiol group (SH), a cycloalkyl group containing from 3 to 20 carbon atoms, said cycloalkyl optionally substituted with at least one group chosen from a hydroxyl group (OH) or a thiol group (SH), an alkoxy group containing from 1 to 2 carbon atoms, said alkoxy group optionally substituted with at least one group chosen from a hydroxyl group (OH) or a thiol group (SH), an aryl group containing from 6 to 12 carbon atoms, said aryl group optionally substituted with at least one group chosen from a hydroxyl group (OH) or a thiol group (SH), or a radical —(O)_{p′}—Si(R₁)_{m′}(R₂)_{n′};
A is independently chosen from saturated divalent hydrocarbon-based radicals containing from 1 to 2 carbon atoms,
X is chosen from a hydrogen atom or a saturated divalent hydrocarbon-based radical containing from 1 to 2 carbon atoms;
m independently denotes an integer ranging from 1 to 3;
n independently denotes an integer ranging from 0 to 2;
m′ independently denotes an integer ranging from 1 to 3;
n′ independently denotes an integer ranging from 0 to 2;
p is an integer equal to 0, 1, 2, or 3;
p′ is an integer equal to 0 or 1;
k is an integer equal to 0, 1, 2, or 3;
q is an integer equal to 0 or 1;
j independently denotes an integer equal to 0 or 1;
t is an integer equal to 0 or 1;
y is an integer ranging from 0 to 10;
z is an integer ranging from 0 to 500;
m+n = 3;
m′+n′ = 3;
x+z ranges from 0 to 500; and
x+y+z ≥ 4;
with the proviso that:
if X represents a hydrogen atom, then t = 0 and p = 1, and
if p = 0, then t = 1 and q = 1, and that at least one of the radicals R₁ or R₃ denotes a hydroxyl radical or an alkoxy radical containing from 1 to 2 carbon atoms;
wherein composition (A) and/or composition (B) optionally further comprise at least one coloring agent chosen from pigments, direct dyes, or mixtures thereof.

41. The process according to claim 40, comprising a step of treating the keratin fibers by applying to said keratin fibers a cosmetic composition (G) comprising at least one alkoxysilane chosen from the compounds of formula (XVIII) below, oligomers thereof, or mixtures thereof: 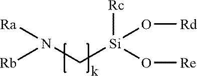
in which:
Ra and Rb are independently chosen from a hydrogen atom, alkyl groups containing from 1 to 20 carbon atoms, cycloalkyl groups containing from 3 to 20 carbon atoms; aryl groups containing from 6 to 12 carbon atoms, or aminoalkyl groups containing from 1 to 20 carbon atoms;
Rc represents a hydrogen atom, an alkyl group containing from 1 to 20 carbon atoms, or an alkoxy group containing from 1 to 10 carbon atoms
Rd and Re are independently chosen from alkyl groups containing from 1 to 10 carbon atoms; and
k denotes an integer ranging from 0 to 5;
wherein composition (G) is applied to the keratin fibers before the application of composition (F).

42. The process according to claim 41, wherein:
Ra and Rb, which are identical, represent a hydrogen atom;
Rc represents an alkyl group containing from 1 to 10 carbon atoms or an alkoxy group containing from 1 to 4 carbon atoms; and
k denotes an integer ranging from 1 to 3.

43. The process according to claim 41, wherein the at least one alkoxysilane of formula (XVIII) is chosen from 3-aminopropyltriethoxysilane (APTES), 3-aminopropylmethyldiethoxysilane (APMDES), N-cyclohexylaminomethyltriethoxysilane, or mixtures thereof.

44. The process according to claim 41, wherein a washing, rinsing, drying, and/or draining step is performed after applying composition (G) to the keratin fibers and before applying composition (F) to the keratin fibers.
